(19) United States
(12) Patent Application Publication
(10) Pub. No.: US 2025/0046427 A1
(43) Pub. Date: Feb. 6, 2025

(54) AUTOMATED ASSESSMENT OF MEDICAL CONDITIONS

(71) Applicant: Nelson M. HANDAL, Dothan, AL (US)

(72) Inventor: Nelson M. HANDAL, Dothan, AL (US)

(21) Appl. No.: 18/603,621

(22) Filed: Mar. 13, 2024

Related U.S. Application Data
(63) Continuation of application No. 16/634,053, filed on Jan. 24, 2020, now abandoned.
(60) Provisional application No. 62/537,880, filed on Jul. 27, 2017.

(51) Int. Cl. G16H 20/70; G09B 7/02; G09B 7/06; G09B 19/00

(52) U.S. Cl. CPC G16H 20/70; G09B 7/02; G09B 7/06; G09B 19/00

(57) ABSTRACT

Systems and methods are provided for patient evaluation. The systems may include a mobile device and a central server. The mobile device can conduct an automated assessment that detects at least one of traumatic brain injury or concussion in a patient. The automated assessment can include in part displaying a clinical condition questionnaire and receiving responses to the clinical condition questionnaire from the patient. The mobile device can provide a record of the automated assessment of the patient to the central server. The central server can receive the record of the automated assessment of the patient, receive a request to determine a clinical state of a patient, diagnose the clinical state of the patient based on the received record of the automated assessment and stored records of prior automated assessments, and provide an indication of the clinical state of the patient.

### CROSS REFERENCE TO RELATED APPLICATION

This application claims the benefit of U.S. Provisional Application No. 62/537,880, filed Jul. 27, 2017, which is incorporated here by reference in its entirety.

### TECHNICAL FIELD

The disclosed embodiments concern systems and methods for automated assessment and diagnosis of patient clinical states. The disclosed embodiments also concern a visual programming language for constructing questionnaires for automated assessment and diagnosis of patient clinical states.

### SUMMARY

The disclosed embodiments include an automated real-time warning system. This system can be configured to alert healthcare providers or emergency services when a patient taking an automated diagnostic exam was determined to pose a threat to themselves or others. This automated real-time warning system can include at least one processor and at least one non-transitory computer readable medium. This computer readable medium can contain instructions that, when executed by the at least one processor, cause the automated real-time warning system to perform operations. These operations can include receiving, from a remote device, a request to perform an automated assessment of a patient. These operations can further include performing an automated assessment of the patient to determine a potential clinical state of the patient. And the operations can also include sending a message indicating a potential for harm associated with the patient based on the potential clinical state of the patient.

The disclosed embodiments include a method for evaluating traumatic brain injury. This method can enable rapid evaluation of potentially injured individual by untrained individuals in a non-clinical setting. For example, the method can be used to evaluate individuals suspected of having a concussion or other traumatic brain injury. Embodiments of this method of patient evaluation can include performing an automated assessment that detects at least one of traumatic brain injury or concussion. This automated assessment can be performed on the patient using a mobile device. The automated assessment can include, in part, displaying at least one of traumatic brain injury questionnaire or a concussion questionnaire. The automated assessment can also include receiving responses to the at least one of traumatic brain injury questionnaire or a concussion questionnaire. And the method can further include providing, using the mobile device, an indication of a clinical state of the patient based on the automated assessment.

The disclosed embodiments also include a system for evaluating traumatic brain injury. This system can include a mobile device including at least one processor and at least one non-transitory memory. The non-transitory memory can contain instructions that when executed by the processor cause the mobile device to perform mobile device operations. These mobile device operations can include conducting an automated assessment that detects at least one of traumatic brain injury or concussion. This automated assessment can include in part displaying a clinical condition questionnaire and receiving responses to the questionnaire from the patient. The mobile device operations can further include providing a record of the automated assessment of the patient to a central server.

The central server can also include at least one processor and at least one non-transitory memory containing instructions. When executed by the processor of the central server, these instructions can cause the central server to perform central server operations. These server operations can include receiving the record of the clinical assessment of the patient. They can further include receiving a request to determine a clinical state of a patient and diagnose a clinical state of the patient based on the received record of the automated assessment and stored records of automated assessments. The server operations can also include providing an indication of the clinical state of the patient.

The disclosed embodiments also include another system for evaluating traumatic brain injury. This system can include at least one processor and at least one non-transitory computer readable medium containing instructions. When executed by the at least one processor, the instructions can cause the automated assessment system to perform operations. The operations can include receiving, from a remote device, a first request to perform an automated assessment of a patient. The operations can further include performing a first automated assessment of the patient using a first questionnaire to determine a potential clinical state of the patient in response to the first request. The operations can additionally include receiving at least one second request to perform at least one follow-up automated assessment of the patient. This at least one second request can be received from the remote device after determining the potential clinical state of the patient. The operation can also include performing at least one follow-up automated assessment of the patient using at least one second questionnaire in response to the at least one second request.

The disclosed embodiments include an automated system for tracking a patient's condition. This automated assessment system can include at least one processor and at least one non-transitory computer readable medium containing instructions. When executed by the at least one processor, these instructions can cause the automated assessment system to perform operations. The operations can include receiving, from a remote device, a request to perform an automated assessment of a patient. The operations can also include performing an automated assessment of the patient to determine a potential clinical state of the patient. The automated assessment can include communicating with the remote device to display a general questionnaire and to receive responses to the general questionnaire. The automated assessment can also include selecting at least one clinical state-specific questionnaire based on the responses to the general questionnaire. The automated assessment can further include communicating with the remote device to display the at least one clinical state-specific questionnaire and to receive responses to the at least one clinical state-specific questionnaires. And the automated assessment can include determining the potential clinical state of the patient based in part on the responses to the general questionnaire and the at least one clinical state-specific questionnaires.

The disclosed embodiments include a system configured to display a specific graphical user interface to the clinician. This system can include a computing device including at least one processor and at least one memory storing instructions. When executed by the at least one processor, the instructions can cause the computing device to provide a graphical user interface for displaying diagnostic information. The graphical user interface can include a display linked to a medical record of a patient that depicts severity scores. The severity scores can correspond to responses to questions in one or more diagnostic questionnaires for at least one of a plurality of assessments. The display can also include a severity axis and a question axis. Indicators on the severity axis can correspond to the severity scores and can be arranged from least severity to greatest severity. Indicators on the question axis can corresponding to the questions in the one or more diagnostic questionnaires and can be arranged into multiple portions corresponding to multiple clinical states. Each of the multiple portions can be labeled on the display. Divisions between the multiple portions can be labeled on the display.

The graphical user interface can further include display controls arranged below the display that control representation of the severity scores and selection of the at least one of the plurality of assessments for the display.

The graphical user interface can also include a clinical state panel linked to the medical record of the patient that includes diagnostic elements and editing controls. Each diagnostic element can include an indication of a diagnosis for one of the multiple clinical states, a severity of the diagnosis, and a status of the diagnosis. The editing controls can enable editing of the diagnosis panel to update the medical record of the patient.

The disclosed embodiments include a system configured to run a visual programming environment for creating questionnaires. This visual programming system for creating questionnaires can include at least one processor and at least one non-transitory memory containing instructions. When executed by the at least one processor, the instructions can cause the visual programming system to perform operations. The operations can include generating, based on user input, a symbolic representation of a questionnaire comprising question objects, answer objects, and line objects that connect other objects. The operations can also include converting, in response to user input, the symbolic representation of a questionnaire into at least one data structure representing the questionnaire, the data structure usable by an execution engine to automatically perform the questionnaire.

It is to be understood that both the foregoing general description and the following detailed description are exemplary and explanatory only and are not restrictive of the disclosed embodiments, as claimed.

### BRIEF DESCRIPTION OF THE DRAWINGS

The drawings are not necessarily to scale or exhaustive. Instead, emphasis is generally placed upon illustrating the principles of the inventions described herein. The accompanying drawings, which are incorporated in and constitute a part of this specification, illustrate several embodiments consistent with the disclosure and together with the description, serve to explain the principles of the disclosure. In the drawings:

FIG. 1 depicts a schematic of an exemplary system for automated assessment of medical conditions.

FIG. 2 depicts an exemplary automated early warning process that uses an automated assessment of medical conditions.

FIGS. 3A and 3B depict details of exemplary automated assessment processes.

FIG. 4 depicts an exemplary automatic traumatic brain injury and/or concussion assessment process performed using a mobile device.

FIG. 5 depicts a system for exemplary automatic traumatic brain injury and/or concussion assessment using a central server.

FIG. 6 depicts an exemplary process for automatic patient assessment.

FIG. 7 depicts an exemplary graphical user interface for displaying patient diagnostic information.

FIG. 8 depicts an exemplary process for generating questionnaires using a visual programming language.

FIG. 9 depicts an exemplary algorithm blueprint constructed using a visual programming language.

FIGS. 10A-10E depict exemplary interfaces for modifying objects.

FIGS. 11A-11D depict exemplary questionnaire question types.

FIGS. 12A and 12B depict exemplary complaint questionnaires.

FIG. 13 depicts a schematic of an exemplary computing device for performing the envisioned systems and methods.

### DETAILED DESCRIPTION

Reference will now be made in detail to the disclosed embodiments, examples of which are illustrated in the accompanying drawings. Wherever convenient, the same reference numbers will be used throughout the drawings to refer to the same or like parts.

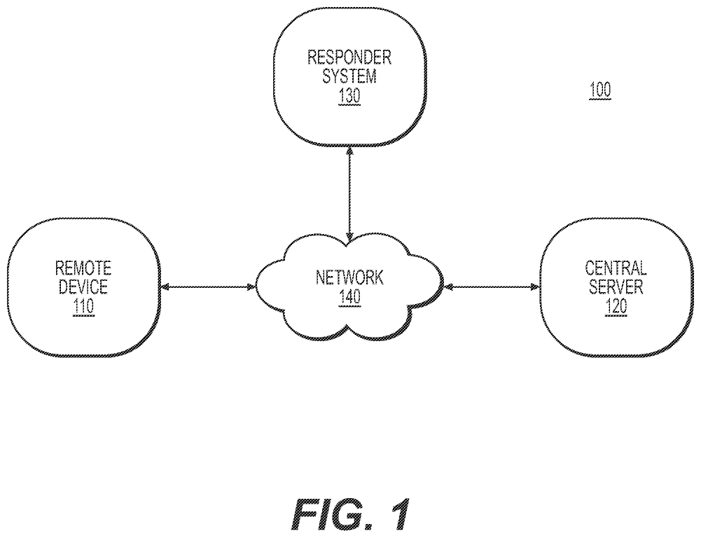

FIG. 1 depicts a schematic of an exemplary system for automated assessment of medical conditions (system 100), consistent with disclosed embodiments. Components of system 100 can include remote device 110, central server 120, and responder system 130. System 100 can be configured to allow users to provide background medical information in advance of a clinical visit, and to perform follow-up evaluations without additional clinical visits. In some embodiments, system 100 can be configured to perform complex and targeted assessments impossible to realize in a clinical setting for reasons of time, distraction, and human error (e.g., forgetting to ask certain questions, lacking available information to answer certain questions). In this manner, system 100 describes a technical solution that improves upon traditional questionnaire systems that rely upon healthcare providers to perform clinical assessments to determine the clinical condition of the user or beneficiary.

Remote device 110 can include one or more computing devices, such as wearable computing devices (e.g., smartwatches, fitness trackers, etc.), smartphones, tablets, laptops, desktops, workstations, or servers consistent with disclosed embodiments. Remote device 110 can be operated or controlled by a user (not shown). Remote device 110 can comprise computer code, instructions, and/or stored data, and can execute the computer code and/or instructions using the stored data to perform aspects of the disclosed systems and methods. For example, a user can interact with remote device 110 to complete a questionnaire. The user can complete this questionnaire on her own behalf, or on behalf of another person (a beneficiary). As an additional example, remote device 110 can be configured to determine a potential clinical state of the user or the beneficiary, and to provide an indication of that potential clinical state. This indication of the potential clinical state can be provided to the user, the beneficiary, another component of system 100 (e.g., central server 120 or responder system 130), or another system.

Central server 120 can include one or more computing devices, such as servers, workstations, desktop computers, or special-purpose computing devices, consistent with disclosed embodiments. Central Server 120 may be standalone, or it may be part of a subsystem, which may be part of a larger system. Central Server 120 can comprise computer code, instructions, and/or stored data, and can execute the computer code and/or instructions using the stored data to perform aspects of the disclosed systems and methods. For example, a user can interact with remote device 110 to complete a questionnaire. As an additional example, central server 120 can be configured to determine a potential clinical state of the user the beneficiary, and to provide an indication of that potential clinical state. As a further example, central server 120 can be configured to provide a message indicating a potential for harm. This message can concern a potential of the user or the beneficiary to harm themselves or others. In some aspects, the indication of the potential clinical state and/or the message can be provided to the user, the beneficiary, another component of system 100 (e.g., remote device 110 or responder system 130), or another system.

Responder system 130 can include one or more computing devices, such as servers, workstations, desktop computers, or special-purpose computing devices, consistent with disclosed embodiments. Responder system 130 can be standalone, or it may be part of a subsystem, which may be part of a larger system. Responder system 130 can comprise computer code, instructions, and/or stored data, and can execute the computer code and/or instructions using the stored data to perform aspects of the disclosed systems and methods. For example, in some embodiments responder system 130 can be associated with a health or safety organization (e.g., a public or private clinic or hospital, a suicide-prevention organization, a police department, a fire department, an emergency medical services department). In some aspects, the health or safety organization can have a pre-existing relationship with the user and/or the beneficiary. For example, the health or safety organization can employ or be associated with a primary care physician of the user or the beneficiary, or with a specialist (such as a mental health specialist) currently or previously responsible for treatment of the user or the beneficiary.

Network 140 may be configured to provide communications between components of FIG. 1. For example, network 140 may be any type of network (including infrastructure) that provides communications, exchanges information, and/or facilitates the exchange of information, such as the Internet, a Local Area Network, or other suitable connection(s) that enables system 100 to send and receive information between the components of system 100.

As would be appreciated by one of ordinary skill in the art, the particular division of functions depicted in FIG. 1 is not intended to be limiting. In some embodiments, one system can be configured to perform the functions of one or more components of system 100. For example, one system can perform the functionality of one or more of central server 120 and responder system 130. In some embodiments, multiple systems can be configured to perform the functions of a component of system 100. For example, multiple systems can perform the functions of central server 120. In this example, a first system can conduct an assessment, while a second system sends a message to responder system 130. Additional rearrangements and distributions of functionality would be apparent to one of skill in the art.

System 100 can be configured as an automated early warning system, consistent with disclosed embodiments. This automated early warning system could be configured to alert a health or safety organization when a patient taking an automated diagnostic exam was determined to pose a threat to themselves or others. In some embodiments, this determination can be made before the patient has completed the questionnaire. A message can then be provided immediately. This improves upon systems requiring users to provide completed questionnaires to medical professionals for evaluation. Such systems are deficient because they cannot address the potential for immediate harm-the medical professionals may evaluate the questionnaire long after it is completed by the user (and potentially after the predicted harm has occurred).

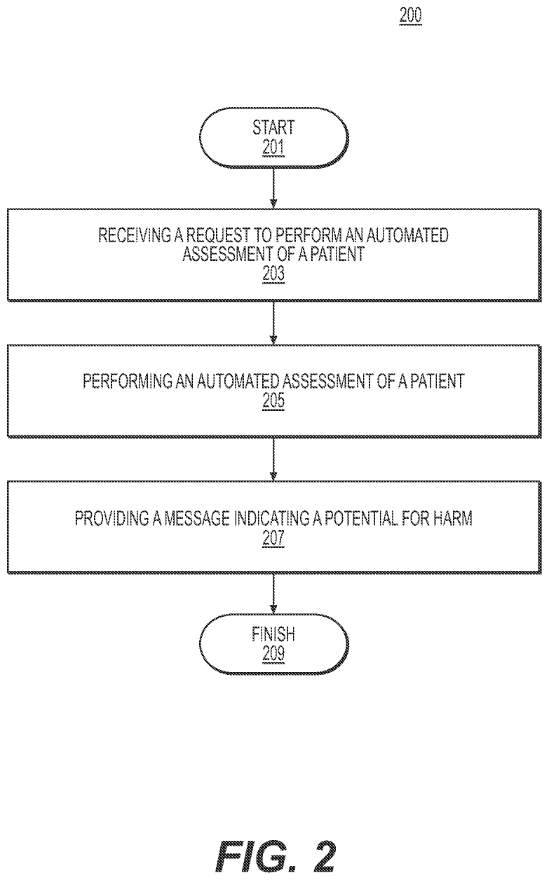

FIG. 2 depicts an exemplary automated early warning process (process 200) that uses an automated assessment of medical conditions, consistent with disclosed embodiments. In some embodiments, as described in greater detail below, process 200 includes the steps of receiving a request to perform an automated patient assessment, performing the automated assessment, and providing a message indicating a potential for harm. In various aspects, process 200 can be performed using components of system 100.

After starting at step 201, process 200 can include receiving a request to perform an automated assessment of a patient. In some embodiments, this request can be received by central server 120. This request can be received from remote device 110. For example, a user can interact with remote device 110, causing remote device 110 to send the request to central server 120. Alternatively, or additionally this request can be received by central server 120 from another system (e.g., another computing device). For example, a healthcare provider can interact with another computing device to cause that computing device to send a request to central server 120. In some embodiments, the request can include authentication and authorization information, such as a password or username.

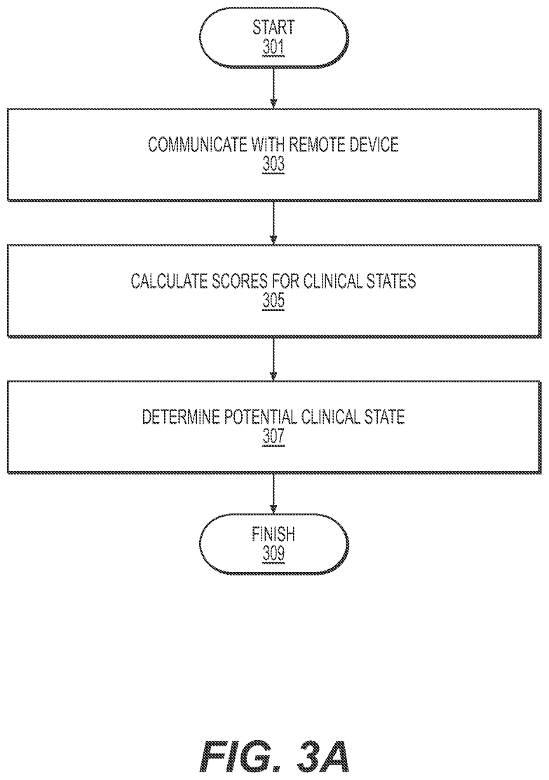

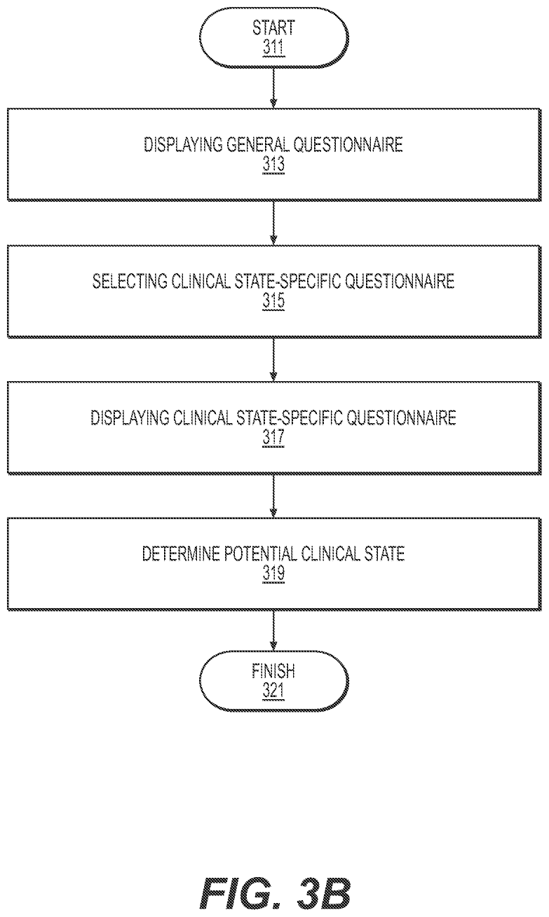

In step 205, central server 120 can perform an automated assessment of the patient, consistent with disclosed embodiments. As described in greater detail below with regard to FIG. 3, this automated assessment can involve providing a questionnaire including questions relevant to one or more clinical conditions and receiving user responses to the questions.

In some embodiments, central server 120 can be configured to determine the potential clinical state of the user or beneficiary based in part on the assessment and in part on other information. For example, central server 120 can be configured to access a medical record of the user or beneficiary following the request to perform the automated assessment and prior to determining the potential clinical state of the user or beneficiary. Central server 120 can be configured to determine the potential clinical state of the user or beneficiary based in part on the information contained in the medical record. For example, when the medical records indicate that the user or beneficiary was previously diagnosed with depression, central server 120 can be configured to lower a threshold for diagnosing depression. Alternatively, central server 120 can be configured to modify a score for a “depression” clinical-state. For example, central server 120 can be configured to increase the score for the “depression” clinical state. In some aspects, lower the threshold and increasing the score can make a diagnosis of a “depression” clinical state more likely.

In various embodiments, central server 120 can be configured to determine the potential clinical state of the user or beneficiary based in part on a comparison to population data. For example, central server 120 can be configured with statistical data describing ranges of possible scores for clinical states. This statistical data can be based on population studies. In some aspects, central server 120 can be configured with thresholds for determining abnormal clinical states based on standard deviations of scores for a representative population of patients. For example, according to methods know in the art, a threshold for classifying individuals as suicidal (or non-suicidal) can be determined based on scores for the “suicidal” clinical state obtained for a representative population of suicidal and non-suicidal individuals. This threshold can depend on an expected loss or conditional risk. Alternatively, the threshold can be based on the variance of the population as a whole. For example, the threshold can be set at a certain quartile, percentile, standard deviation from the mean, or similar measurement.

In step 207, central server 120 can send a message indicating a potential for harm, consistent with disclosed embodiments. In some embodiments, the message can be an automatically generated text message, automated phone call, automatically generated email, or similar message. In some embodiments, the message can be sent during performance of the automated assessment. For example, the message can be sent while the user or beneficiary is completing the questionnaire. In various embodiments, the message can be sent upon satisfaction of some potential harm criterion. For example, central server 120 can be configured to determine whether the user or the beneficiary poses a potential for harm to themselves or others. As an additional example, central server 120 can be configured to receive the responses to questions while the user completes the assessment. As described below with regards to FIGS. 3A and 3B, central server 120 can be configured to calculate scores for clinical conditions based on these responses. In some aspects, the potential harm criterion can comprise a score threshold for one or more clinical conditions. Should a score for a clinical condition exceed this score threshold, central server 120 can be configured to send a message. Central server 120 can be configured to send the message to responder system 130. For example, the clinical state can be “suicidal” and central server 120 can be configured to track a score for the “suicidal” clinical state. Should this score exceed a predetermined threshold, central server 120 can be configured to send a message to responder system 130 indicated that the user or beneficiary may be at risk of self-inflicted harm. In some embodiments, central server 120 can be configured to send this message without informing the user. Alternatively, remote device 120 can be configured to display to the user the message, or another message indicating the risk of self-inflicted harm.

Following step 207, the early warning process described in FIG. 2 can end in step 209. As would be appreciated by one of skill in the art, the particular order and arrangement of steps disclosed in FIG. 2 is not intended to be limiting. For example, steps can be re-arranged or combined without departing from the envisioned embodiments. Steps can be divided into sub-steps that can be performed in a different order, or by different components of system 100. Furthermore, additional steps may be added to, or steps may be removed from, this process without departing from the envisioned embodiments.

FIGS. 3A and 3B depict details of exemplary automated assessment processes, consistent with disclosed embodiments. FIG. 3A describes in greater detail performance of an assessment consistent with the automated early warning process of FIG. 2. This assessment can be performed using components of FIG. 1.

After beginning at step 301, central 120 can communicate with remote device 110 in step 303 to display questions concerning one or more potential clinical states. For example, central server 120 can provide instructions to remote device 110 to display a graphical user interface, according to methods known to one of skill in the art. These instructions can cause remote device 110 to display a questionnaire. This questionnaire can include questions concerning one or more potential clinical states. In this manner, the questionnaire can resemble Patient Health Questionnaire 9, The Hamilton Anxiety Rating Scale, and Social Phobia Inventory. In some embodiments, central server 120 can communicate with remote device 110 to receive responses to the questions. The user can provide these responses, which can be appropriate for the user (if the questionnaire is intended to evaluate the user) or appropriate for the beneficiary (if the question is intended to evaluate the beneficiary).

In step 305, central server 120 can calculate scores for the one or more potential clinical states, consistent with disclosed embodiments. As described in greater detail below, central server 120 can calculate a score for each question for one or more clinical states. This score for the question, for the one or more clinical states, can be based on a weight for the question and a value for the response to the question, for the one or more clinical states.

The weights for questions can be numbers representative of the importance of the questions to diagnosing a clinical state, consistent with disclosed embodiments. The weights can differ between questions, with questions more predictive of a clinical state (e.g., questions with responses greatly affecting the likelihood of the clinical state) having higher weights than questions less predictive of a clinical state (e.g., questions with responses that do not greatly affect the likelihood of the clinical state). Questions can have multiple weights corresponding to different clinical states. These weights can differ depending on how predictive the question is to each clinical state.

The values for responses can be numbers representative of the importance of the responses to diagnosing a clinical state, consistent with disclosed embodiments Similar to the weights for questions, the values for responses can differ between responses to a question. Responses more predictive of a clinical state (e.g., responses greatly affecting the likelihood of the clinical state) can have higher values than responses less predictive of a clinical state (e.g., questions with responses that do not greatly affect the likelihood of the clinical state). Responses can have multiple values corresponding to different diagnoses. These values can differ depending on how predictive the response is to each diagnosis.

The score for the question can be a function of the weight of the question and the value of the response, consistent with disclosed embodiments. For example, the score for the question can be the product of the weight for the question and the value of the response. The score for a clinical state can be a function of the scores for the questions associated with the clinical state. For example, the score for a clinical state can be the sum of the scores for the questions.

Central server 120 can determine a potential clinical state of the patient based at least in part on the score for the clinical state, consistent with disclosed embodiments. In some embodiments, system 100 can be configured to automatically diagnose the potential clinical state of the patient based on the score for the clinical state. For example, central server 120 can be configured with predetermined thresholds. The predetermined thresholds can differ between one or more clinical states. Alternatively, or additionally, the predetermined thresholds can be the same for one or more clinical states. Central server 120 can be configured to diagnose a user as having a clinical state when the score for the clinical state exceeds the threshold for the clinical state.

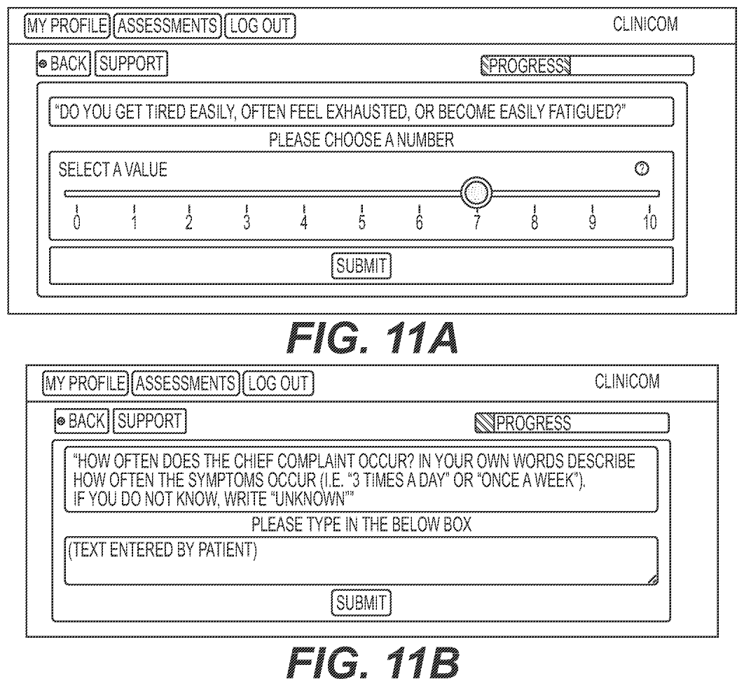

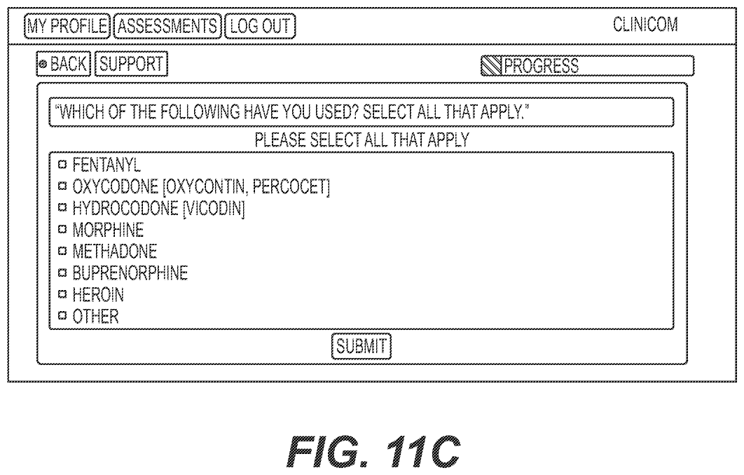

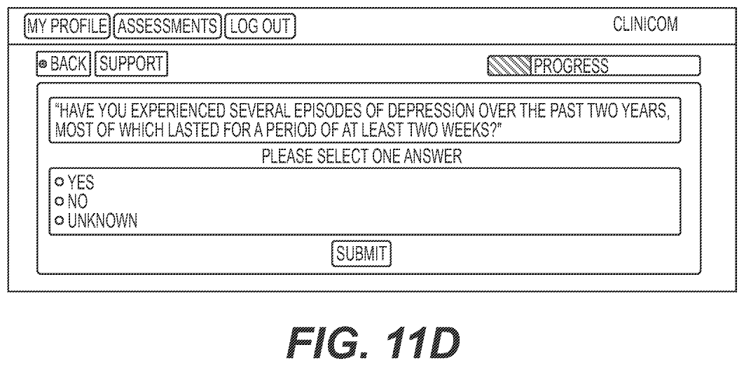

In some embodiments, responses to the questions can include sliders, text boxes, check boxes, and radio buttons. For example, the response can be selected with a slider, as shown in FIG. 11A. In this example, in some aspects, the slider can select the value (e.g., when the slider is set to 7, as shown, the value for the response can be 7). In various aspects, the slider can select the value in a non-linear fashion (e.g., slider settings of 0-3 can map to a value of 0, slider settings of 4-7 can map to values of 1-3, and slider settings of 8-10 can map to a value of 4). As an additional example, the response can be a text box, as shown in FIG. 11B. In some embodiments, regular expressions and/or natural language processing can be used to extract features from the entered text. In some aspects, the entered text can be recorded for subsequent review by a clinician. For example, the entered text can be entered into a medical record of the user or beneficiary.

As a further example, the response can be the selection of one or more check boxes, as shown in FIG. 11C. In some aspects, each check box can contribute to the value for the response. For example, when the response comprises selecting multiple check boxes, the weight for the response can be a function (e.g., a sum) of the contributions for those check boxes. In some embodiments, the check boxes can have equal contributions. For example, the weight for the response can simply be the number of selected checkboxes. In various embodiments, the check boxes can have unequal contributions. For example, with respect to the question shown in FIG. 11C, the check box for heroin can contribute more to the value of the response than the check box for hydrocodone.

As a further example, the response can be the selection of one of a set of mutually exclusive radio buttons, as shown in FIG. 11D. Three potential responses can be “yes,” “no,” and “unknown.” The value for the response can depend on the selected radio button. As a non-limiting example, a value of 10 can be associated with the response “yes” and a value of 0 can be associated with the responses “no” and “unknown.”

In some embodiments, central server 120 can be configured to determine the potential clinical state of the patient based in part on prior scores for the user or beneficiary. For example, central server 120 can be configured to compare the present score for a clinical state and one or more previously calculated scores for the clinical state. For example, central server 120 can be configured to diagnose a clinical state, or the absence of a previously diagnosed clinical state, based on a change between the present score and the one or more previously calculated scores for the clinical state. As an additional example, central server 120 can be configured to diagnose a clinical state or the absence of a previously diagnosed clinical state, based on a function of the one or more previously calculated scores for the clinical state. For example, this function can be an average, or a trend line determined from the calculated scores for the present clinical state and the one or more previous clinical states.

Following step 307, the assessment process described in FIG. 3A can end in step 309. As would be appreciated by one of skill in the art, the particular order and arrangement of steps disclosed in FIG. 3A is not intended to be limiting. For example, steps can be re-arranged or combined without departing from the envisioned embodiments. Steps can be divided into sub-steps that can be performed in a different order, or by different components of system 100. Furthermore, additional steps may be added to, or steps may be removed from, this process without departing from the envisioned embodiments.

As shown in FIG. 3B, central server 120 can be configured to determine the potential clinical state of the user or beneficiary with a general questionnaire and a clinical state-specific questionnaire. The clinical state-specific questionnaire can include questions tailored to a specific clinical state. By dividing the assessment between a general questionnaire and a state specific questionnaire, system 100 can focus on relevant potential clinical conditions and avoid burdening users with the need to answer irrelevant questions.

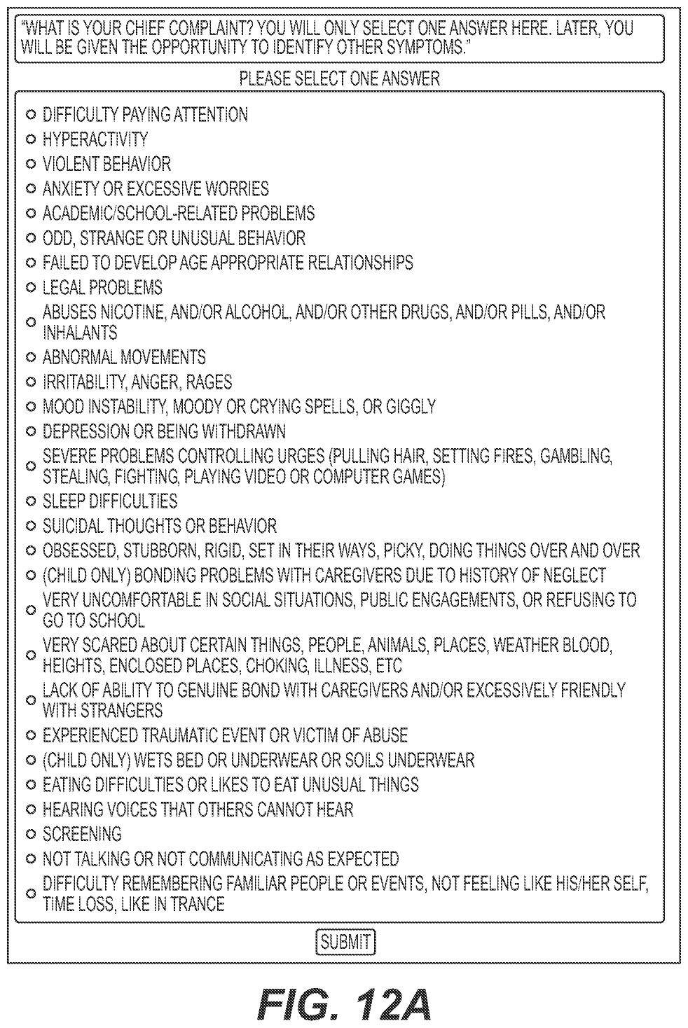

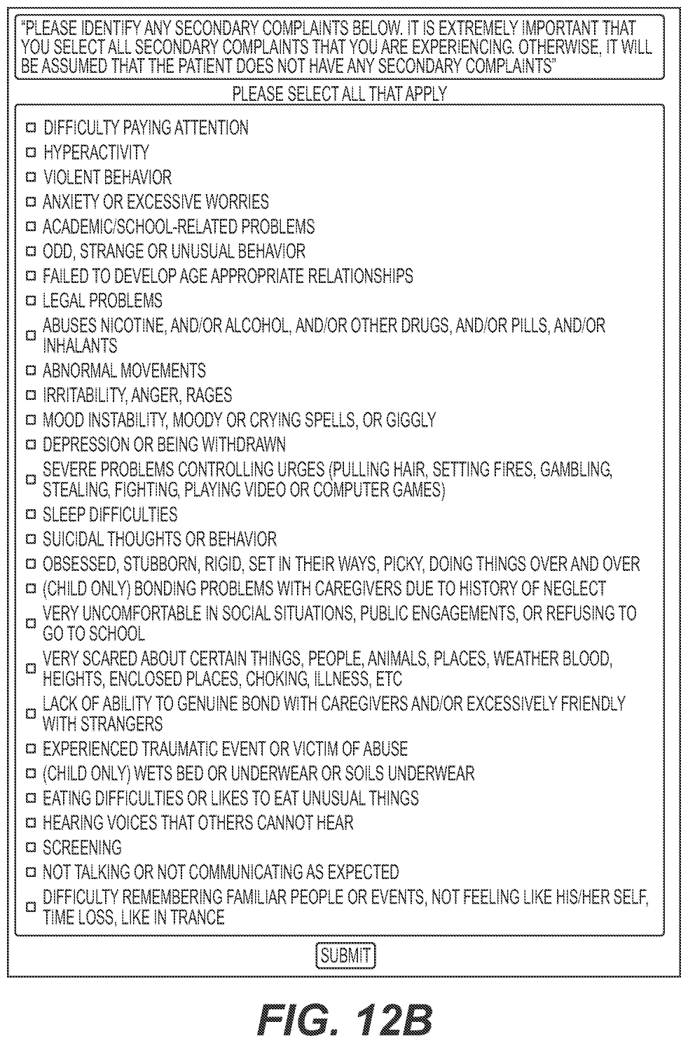

After starting in step 311, system 100 can be configured to display a general questionnaire in step 313, consistent with disclosed embodiments. For example, central server 120 can be configured to send instructions to remote device 110 to display the general questionnaire to the user. Examples of this general questionnaire are provided in FIG. 12A and FIG. 12B. As shown these figures, the general questionnaire can include questions screening the user or the beneficiary for many possible clinical states. For example, as shown in FIG. 12A and FIG. 12B, a wide range of symptoms are presented, concerning clinical states ranging from hyperactivity to depression to traumatic brain injury. The user can select one or more of these checkboxes to indicate a primary complaint and one or more secondary complaints. The indicated complaints can be complaints of the user or the beneficiary, depending on whether the user or the beneficiary is being assessed. In various aspects, central server 120 can be configured to receive responses to the general questionnaire.

In step 315, based on the response to the general questionnaire, central server 120 can be configured to identify one or more clinical conditions as potential clinical states of the user or the beneficiary. In some embodiments, this determination can be based upon a predetermined relationship between the responses to the general questionnaire and the potential clinical states. For example, when the user selects the checkbox for “hyperactivity,” central server 120 can be configured to select a clinical state-specific questionnaire for ADHD for further assessing the user or beneficiary for ADHD. Similarly, when the user selects “depression or being withdrawn” for a secondary complaint, central server 120 can be configured to select a clinical state-specific questionnaire for depression for further assessing the user or beneficiary for depression.

In step 317, following selection of one or more clinical state-specific questionnaires, system 100 can be configured to display the one or more clinical state-specific questionnaires to the user. For example, central server 120 can be configured to send instructions to remote device 110 to display the one or more clinical state-specific questionnaires. Examples of state specific questionnaire questions include those depicted in FIG. 11A-11D and discussed above with regard to FIG. 3A. In some embodiments, central server 120 can be configured to display the state-specific questionnaires separately. For example, central server 120 can provide the first questionnaire for a first clinical state and then provide the second questionnaire for the second clinical state once the first questionnaire is complete. Alternatively, according to this example, central server 120 can be configured to provide the questions for the first and second clinical questionnaires together. The questions for the first and second clinical questionnaires can be intermingled. In some embodiments, multiple weights and values for responses can be assigned to the questions for each questionnaire. Questions common to multiple questionnaires can then be displayed only once. In various aspects, central server 120 can be configured to receive responses to the general questionnaire.

In step 319, central server 120 can be configured to determine the potential clinical state of the patient based in part on the responses to the at least one clinical state-specific questionnaire. In some embodiments, central server 120 can be configured to calculate scores for clinical states, as described above with regards to FIG. 3A. For example, central server 120 can be configured to associate weights with the clinical-state specific questions, and values with the responses to the clinical-state specific questions. Furthermore, central server 120 can be configured to calculate the contribution of each question to an overall clinical state-specific score. In some embodiments, by comparing this overall score to a predetermined threshold, central server 120 can be configured to diagnosis the user as having (or not having) the clinical state.

Following step 319, the assessment process described in FIG. 3A can end in step 321. As would be appreciated by one of skill in the art, the particular order and arrangement of steps disclosed in FIG. 3B is not intended to be limiting. For example, steps can be re-arranged or combined without departing from the envisioned embodiments. Steps can be divided into sub-steps that can be performed in a different order, or by different components of system 100. Furthermore, additional steps may be added to, or steps may be removed from, this process without departing from the envisioned embodiments.

System 100 can be configured to enable evaluation of the clinical condition of a potentially injured user or beneficiary. For example, the user can communicate with remote device 110 to complete a questionnaire for evaluating suspected concussions or other traumatic brain injuries. According to the disclosed systems and methods, this evaluation can be accomplished by untrained individuals in non-clinical settings, such as recreational sports settings, wilderness areas, armed forces training operations, or other venues traditionally lacking clinically trained individuals competent to determine a clinical condition of the user or beneficiary. In some aspects, system 100 can enable evaluation of the clinical condition of the potentially injured user or beneficiary without using specialized equipment. As a non-limiting example, remote device 110 can comprise a smartphone configured with a downloaded application that performs the envisioned systems and methods. In this manner, system 100 describes a technical solution that allows untrained individuals in non-clinical settings to determine the clinical condition of a potentially injured user or beneficiary. This increases the likelihood that injured users or beneficiaries will be identified and treated, and could allow emergency responders advance notice of the potentially injured user or beneficiary's condition.

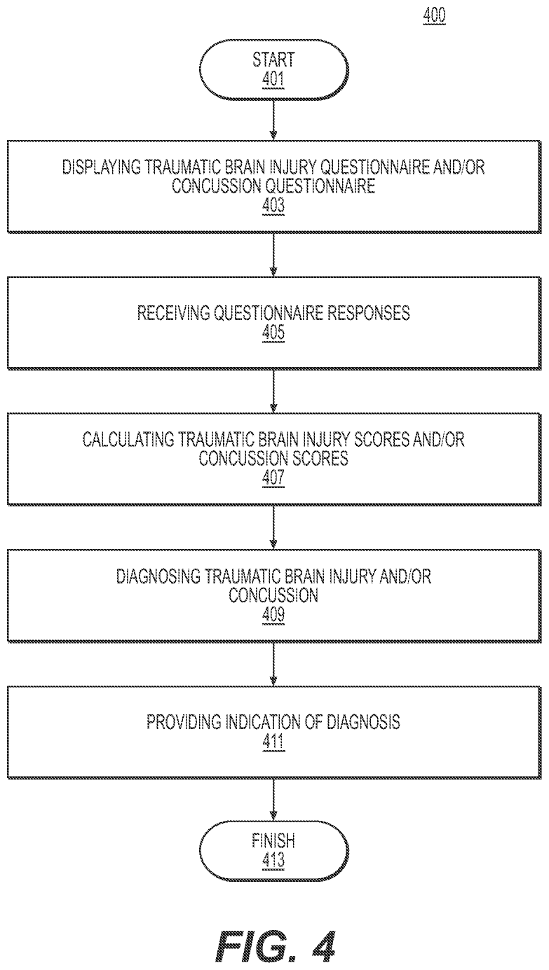

FIG. 4 depicts an exemplary automatic traumatic brain injury and/or concussion assessment process (process 400) performed using a mobile device, consistent with disclosed embodiments. The mobile device can include a fitness tracker, smartwatch, smartphone, tablet, laptop, or similar device. In some embodiments, process 400 can include the steps of displaying a traumatic brain injury and/or concussion questionnaire, receiving questionnaire responses, calculating traumatic brain injury scores and/or concussion scores, determining a potential clinical state, and providing an indication of the potential clinical state. According to process 400, components of system 100 can be configured to enable evaluation of the clinical condition of a potentially injured user or beneficiary. For example, remote device 110 can be the mobile device.

In some embodiment, process 400 can be performed in response to an event capable of causing at least one of traumatic brain injury or concussion. For example, a football coach can use a mobile device to perform the automated assessment during a game for a player that received a hit to the head. As an additional example, a medic can use a mobile device to perform the automated assessment during a military campaign for a soldier exposed to an explosive blast. As a further example, a police officer can use a mobile device to perform the automated assessment at the scene of a car accident for a passenger of the car. As demonstrated by these examples, the event capable of causing at least one of traumatic brain injury or concussion includes events such as a hit to the head, explosive blast, or car accident. As would be appreciated by one of skill in the art, these events are not intended to be limiting. Many other events would be recognized as capable of causing at least one of traumatic brain injury or concussion. In some embodiments, the event may be recent. For example, the event may have occurred less than 5 minutes, 15 minutes, 30 minutes, an hour, two hours, a day, or a week before performance of the automated assessment of the user or beneficiary.

After starting at step 401, the mobile device can be configured to display a traumatic brain injury questionnaire and/or concussion questionnaire in step 403. For example, the mobile device can be configured to execute an application for assessing people for traumatic brain injuries and/or concussions. This assessment can include display a traumatic brain injury questionnaire and/or concussion questionnaire. In some aspects, the questionnaire can be the Awareness Questionnaire and Warrior Administered Retrospective Causality Assessment Tool, or a similar questionnaire. For example, the mobile device can be configured to display a questionnaire based on The Awareness Questionnaire and Warrior Administered Retrospective Causality Assessment Tool. The questionnaire can be displayed on a screen of the mobile device. As non-limiting examples, questionnaire questions can be formatted as shown in FIGS. 11A-11D, 12A and 12B. The questionnaire can be displayed in response to a request received from another system (e.g., central server 120) or in response to instructions received from the user (e.g., through an input/output device of the mobile device, such as a touchscreen or keyboard). As described above with regards to FIGS. 2, 3A and 3B, this questionnaire can comprise questions concerning the clinical state of traumatic brain injury or concussion.

In step 405, the mobile device can be configured to receive questionnaire responses. These questionnaire responses can be received from the user. The responses can concern the user (if the user is being evaluated) or another beneficiary (if that beneficiary is being evaluated). For example, the mobile device can receive the questionnaire responses through an input/output device of the mobile device, such as a touchscreen or keyboard. As would be appreciated by one of skill in the art, the displaying of the questionnaire and the receiving of the responses can be intermixed over the same interval of time. For example, the mobile device can be configured to display one of the questions in the questionnaire and then receive a response to that question, before displaying another question. As an additional example, the mobile device can be configured to display multiple questions and then receive responses to these multiple questions, before displaying additional questions.

In step 407, at least one of the mobile device or central server 120 can be configured to calculate traumatic brain injury and/or concussion scores. For example, the mobile device can be configured to execute an application that calculates the traumatic brain injury and/or concussion scores. As an additional example, the mobile device can be configured to provide the responses to central server 120. Central server 120 can then calculate the traumatic brain injury and/or concussion scores. As described above with regards to FIGS. 2, 3A and 3B, the questions in the questionnaire can contribute to an overall clinical score. In some aspects, this contribution can depend on values for the responses to the questions. In various aspects, this contribution can depend on weights for the questions. For example, the contribution can be a function (e.g., a product) of the weights and the values. In some aspects, the calculation of the scores can be an ongoing process, performed as responses are received. In various aspects, the calculation of the scores can be performed upon completion of the questionnaire.

In step 409, at least one of the mobile device or central server 120 can be configured to diagnose a traumatic brain injury and/or concussion based at least in part on the calculated clinical scores. For example, the mobile device can be configured to assign a diagnosis based on clinical scores calculated by the mobile device or received from central server 120. As an additional example, central server 120 can be configured to assign a diagnosis based on clinical scores calculated by central server 120 or received from the mobile device. Central server 120 can be configured to provide this diagnosis to the mobile device. As described above with regard to FIG. 2, 3A and 3B, the diagnosis can depend on predetermined thresholds. System 100 can be configured to diagnose the user or the beneficiary with a traumatic brain injury and/or concussion when the calculated scores exceed the predetermined thresholds. In some aspects, the diagnosis can be performed as responses are received. For example, when a response causes a clinical score to exceed a threshold while displaying the questionnaire, system 110 can be configured to assign a diagnosis prior to completion of the questionnaire. In various aspects, system 110 can be configured to assign a diagnosis upon completion of the questionnaire.

In some embodiments, the potential clinical state of the patient is determined based in part on a comparison of at least one of the traumatic brain injury scores or concussion scores to previously calculated scores. For example, at least one of the mobile device or central server 120 can be configured to compare a traumatic brain injury score or concussion score to a previously calculated traumatic brain injury score or concussion score. In some aspects, the previously calculated scores are for other patients. As discussed above with regard to FIGS. 3A and 3B, the scores for other patients can be used to establish a normal range, or to determine a classification threshold for classifying a traumatic brain injury score or concussion score as indicative of a traumatic brain injury or concussion. In various embodiments, the results of the automated assessment can establish a baseline for future assessments of the patient. For example, system 100 can be configured to perform repeated assessments of the user or beneficiary. The results of these repeated automated assessments can be used to track the condition of the user or beneficiary over time. For example, the repeated assessments can be used to chart a recovery process or indicate a response of the user or beneficiary to a treatment. As an additional example, an automated assessment can be performed at two points to determine whether an event causing a traumatic brain injury or concussion occurred between those two points. For example, an automated assessment can be performed at induction into the armed services and at discharge from the armed services to support a contention that a traumatic brain injury or concussion was service connected.

In various embodiments, process 400 can further include collecting eye-tracking data of the patient using a camera of the mobile device. For example, rather than displaying a question, the mobile device can display a prompt to the user to perform an eye-tracking task (or cause the beneficiary to perform an eye-tracking task) while being recorded by a video camera of the mobile device. Non-limiting examples of diagnostic eye-tracking tasks include smooth-pursuit and saccadic eye-tracking tasks. The mobile device and/or central server 120 can be configured to process the record of the eye-tracking task to collect eye-tracking data. The mobile device and/or central server 120 can be configured to assign a value to the collected eye-tracking data based on predetermined rules. For example, measuring point of gaze or the motion of the eye relative to the head. The eye-tracking task can be assigned a weight, and can contribute to one or more clinical scores based on the weight and the assigned value, consistent with disclosed embodiments.

Similarly, in some embodiments, process 400 can further include obtaining at least one image of the patient using a camera of the mobile device and determining a probable emotional state of the patient based on the at least one image. For example, rather than displaying a question, the mobile device can display a prompt to the user to take a self-portrait (or a portrait of the beneficiary) using a camera of the mobile device. The mobile device and/or central server 120 can be configured to process the self-portrait or portrait to collect emotional state information of the user or beneficiary according to methods known to one of skill in the art. The mobile device and/or central server 120 can be configured to assign a value to the collected emotional state information based on predetermined rules. For example, flat affect or diminished emotional expression. The self-portrait or portrait can be assigned a weight, and can contribute to one or more clinical scores based on the weight and the assigned value, consistent with disclosed embodiments.

Furthermore, in some embodiments, process 400 can further include collecting physiological data. This physiological data can include at least one of heart rate, blood oxygenation, or activity level of the patient. This information can be collected using a sensor device such as a fitness tracker, pulse oximeter, accelerometer, or similar device. This sensor device can comprise the mobile device, or can be in communication with the mobile device, for example using a Bluetooth link, wireless local area network link, near field communication link, or similar link. The mobile device and/or central server 120 can be configured to assign one or more values to the collected physiological data based on predetermined rules. For example, increased heart rate. The physiological data can be assigned one or more weights, and can contribute to one or more clinical scores based on the weights and the assigned values, consistent with disclosed embodiments.

In step 411, following assignment of the diagnosis in step 409, the mobile device can be configured to provide an indication of a clinical state of the patient based on the automated assessment. For example, the mobile device can be configured to display an indication that the user or the beneficiary has or has not been diagnosed with a traumatic brain injury and/or concussion. As an additional example, the mobile device can be configured to display an indicator showing the clinical scores for at least one of the traumatic brain injury or concussion clinical states. The display can indicate the degree to which the clinical scores for the user differ from the scores for the population as a whole. For example, the display can indicate a normal range and the score of the user. The display can be numeric, graphical, or combine numeric and graphical elements.

Following step 411, process 400 can end in step 413. In some embodiments, process 400 can further include, prior to step 413, transporting the patient to a healthcare facility after performing the automated assessment. As would be appreciated by one of skill in the art, the particular order and arrangement of steps disclosed in FIG. 4 is not intended to be limiting. For example, steps can be rearranged or combined without departing from the envisioned embodiments. Steps can be divided into sub-steps that can be performed in a different order, or by different components of system 100. Furthermore, additional steps may be added to, or steps may be removed from, this process without departing from the envisioned embodiments.

System 100 can be configured to use central server 120 as a repository for assessment records. In some embodiments, this configuration can enable healthcare provider to access the assessment records when reviewing a medical history of the user or beneficiary. Using central server 120 as a repository for assessment records can also enable central server 120 to base diagnosis on stored assessment records for multiple individuals, or store assessment records for multiple assessments for the same user. In various embodiments, processing of the assessment responses can be divided between the mobile device and central server 120. The amount of processing performed by the mobile device versus the central server 120 can depend on the computing resources available to the mobile device.

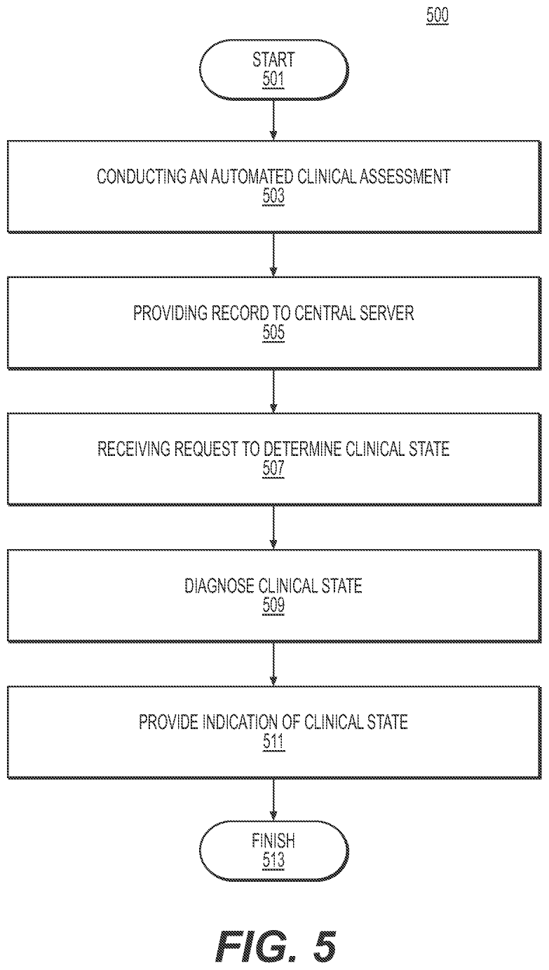

FIG. 5 depicts a process for automatic traumatic brain injury and/or concussion assessment (process 500) performed by an exemplary system that uses central server 120 as a repository for assessment records. In some embodiments, this system can include a mobile device (e.g., a fitness tracker, smartwatch, smartphone, tablet, laptop, or similar device) and central server 120.

After starting at step 501, the mobile device can be configured to conduct an automated assessment in step 503 that detects at least one of traumatic brain injury or concussion, consistent with disclosed embodiments. This assessment can concern the user or the beneficiary. This automated assessment can be performed as described above with regards to FIG. 2, 3A, 3B, and 4. For example, this automated assessment can include displaying, using the mobile device, a clinical condition questionnaire. As an additional example, this automated assessment can include receiving responses to the questionnaire from the user.

In step 505, after performing the automated assessment in step 503, the mobile device can be configured to provide a record of the automated assessment to central server 120, consistent with disclosed embodiments. This record can include at least one of clinical scores for one or more clinical states, contributions of questions to clinical scores for one or more clinical states, weights for questions, values for responses to questions, or indications of the questions provided to the user during the automated assessment.

In step 507, after receiving the automated assessment in step 505, central server 120 can receive a request to determine a clinical state of the user or beneficiary. In some embodiments, central server 120 can receive this request from remote device 110. In various embodiments, central server 120 can receive this request from another computing device. For example, a healthcare provider interacting with another computing device can cause this computing device to provide a request to determine the clinical state of the user or beneficiary to central server 120. As an additional example, a clinician reviewing the automated assessment of the user or beneficiary can interact with a tablet to request that central server 120 determine the clinical state of the user or beneficiary.

In step 509, after receiving the automated assessment in step 507, central server 120 can determine a clinical state of the user or beneficiary based on the received record of the automated assessment and stored records of prior automated assessments. Central server 120 can be configured to determine the clinical state of the user or beneficiary as described above with regard to FIG. 2, 3A, 3B, and 4, consistent with disclosed embodiments. In some embodiments, the stored records can concern the user or the beneficiary. For example, central server 120 can be configured to compare the results of the received automated assessment to stored prior assessments. As a further example, central server 120 can be configured to determine the clinical state of the user or beneficiary based at least in part on a comparison of a clinical score for a “traumatic brain injury” clinical state or a clinical score for a “concussion” clinical state to previously calculated scores.

Central server 120 can be configured to generate a baseline using the stored prior assessments and compare the received assessment to this baseline. In some aspects, increases in clinical scores for clinical states can indicate a worsening clinical state, while decreases in clinical scores for clinical states can indicate an improvement in clinical state. In various aspects, a trend of increasing or decreasing clinical states can indicate a progressively worsening or improving clinical state. In various embodiments, the stored assessments can concern other users or beneficiaries. For example, the stored assessments can concern other patients, some of which can have a traumatic brain injury and/or concussion. Central server 120 can be configured to use the stored assessments to determine a classifying threshold. In some embodiments, central server 120 can be configured to apply the classifying threshold to clinical scores provided in the received automated assessment, or derived from information provided in the received automated assessment, to diagnose the user or beneficiary as having (or not having) a traumatic brain injury or concussion. In various embodiments, the stored assessments can concern a representative sample of other patients, and central server 120 can be configured to determine a diagnostic threshold based on statistics for the clinical scores of this representative population. These statistics can include averages, standard distributions, percentiles (or medians, quartiles, etc.), and other such statistics known to one of skill in the art.

In step 511, at least one of central server 120 or the mobile device can be configured to provide an indication of the diagnosed clinical state. In some embodiments, this indication can be provided to the user by the mobile device. For example, as described above with regards to step 503, the mobile device can be configured to perform an automated clinical assessment. In some aspects, the mobile device can be configured to provide an indication of the diagnosed clinical state of the user or beneficiary to the user during or upon completion of the automated assessment. For example, the mobile device can perform the automated assessment and provide the record of the assessment to central server 120 while also providing an indication of the clinical state to the user. In various aspects, the mobile device can be configured to provide an indication of the diagnosed clinical state to the user following receipt of an indication of the diagnosed clinical state from the central server 120. For example, the mobile device can provide the record of the current assessment to the central server (step 507) and can receive an indication of the diagnosed clinical state from the central server and provide this indication, or a similar indication, to the user (step 511).

In some embodiments, an indication of the diagnosed clinical state can be provided by central server 120 to another computing device. For example, as described above, central server 120 can receive a request to determine a clinical state of the user or beneficiary from another computing device. This computing device can be, for example, operated by a healthcare professional. In step 511, central server 120 can be configured to provide an indication of the diagnosed clinical state to the other computing device. For example, a clinician reviewing the assessment, or the medical records of the user or beneficiary, can request central server diagnose a clinical state of the user or beneficiary, and can then receive an indication of this clinical state from central server 120. In some embodiments, central server 120 can be configured to additionally or alternatively provide an indication of the diagnosed clinical state to the mobile device. For example, the user can interact with the mobile device to perform the automated assessment, the mobile device can provide the record of the automated assessment to central server 120, central server 120 can provide an indication of the diagnosed clinical state of the user or beneficiary to the mobile device, and the mobile device can display this indication to the user.

Following step 511, process 500 can end in step 513. As would be appreciated by one of skill in the art, the particular order and arrangement of steps disclosed in FIG. 5 is not intended to be limiting. For example, steps can be re-arranged or combined without departing from the envisioned embodiments. Steps can be divided into sub-steps that can be performed in a different order, or by different components of system 100. Furthermore, additional steps may be added to, or steps may be removed from, this process without departing from the envisioned embodiments.

System 100 can be configured to perform a process for assessing a patient's condition over time (process 600). According this process, a user or beneficiary can complete an initial diagnostic screening, and can then complete multiple follow-up assessments. Thus system 100 allows for a patient to be tracked over time without having to return to a clinical setting for evaluation.

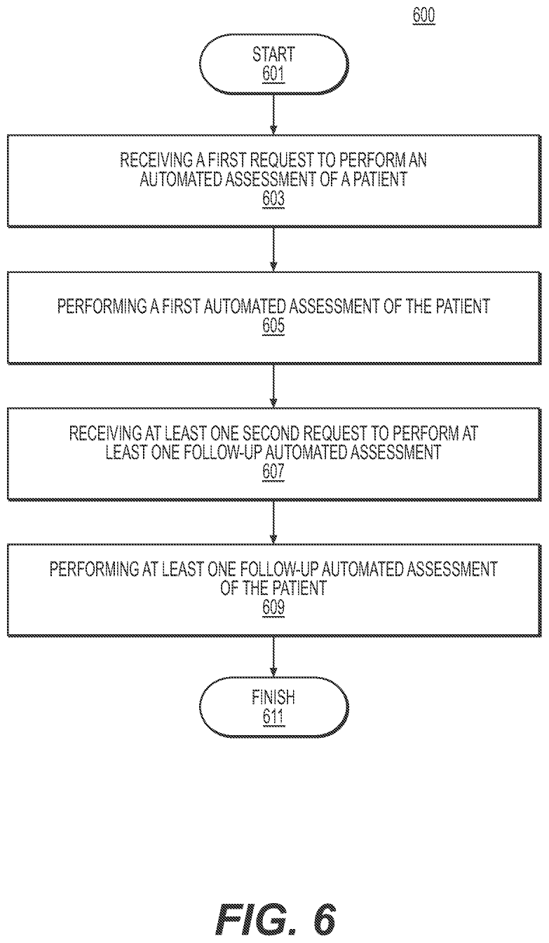

FIG. 6 depicts an exemplary process for automatic patient assessment over time, consistent with disclosed embodiments. In some embodiments, the initial assessment can include the steps of receiving a request to perform an automated assessment, performing a first automated assessment of the patient to determine a potential clinical state of the patient in response to the first request, receiving at least one second request to perform at least one follow-up automated assessment of a patient, and performing at least one follow-up automated assessment of the patient in response to the at least one second request.

After starting at 601, central server 120 can receive a first request to perform an automated assessment of a patient in step 603, consistent with disclosed embodiments. In some embodiments, central server 120 can receive the first request from remote device 110. In various embodiments, central server 120 can receive the first request from another computing device, such as a mobile device, desktop, workstation, or server operated by a healthcare professional. The first request can comprise authentication and authorization credentials identifying the user or beneficiary.

In some embodiments, process 600 can further include providing an invitation to the patient. This invitation can be sent from central server 120 to remote user 110. Alternatively or additionally, this invitation can be sent from another computing device or received by another computing device. The invitation can be configured to enable the user to contact central server 120 to request the assessment. As a non-limiting example, the email can include registration instructions, one or more URLs for the central server 120, and/or a selectable control embedded in the email that causes the computing system of the user to contact central server 120 upon selection of the control, according to methods known to one of skill in the art. The invitation can also include authentication and authorization information. For example, the invitation can specify a username (such as a medical record id number) and a temporary password. As an additional example, upon contacting central server 110, a new user can be instructed to create an account with a user name and password, or enter a user name and password. In some embodiments, the request to perform the first assessment can be received in response to providing the invitation.

In step 605, central server 120 can be configured to perform a first automated assessment of the user or the beneficiary. Central server 120 can be configured to perform the first automated assessment using a first questionnaire, as described above with regard to FIGS. 2, 3A and 3B. For example, as disclosed with regards to FIG. 3B, the first questionnaire can include a general questionnaire. This general questionnaire can include questions concerning a wide range of clinical states. Central server 120 can be configured to use the general questionnaire to identify a chief complaint (and potentially identify at least one secondary complaint). The first questionnaire can also include one or more clinical-state specific questionnaires. Central server 120 can be used to select these questionnaires from a collection of predetermined questionnaires based on the complaint(s) identified by the general questionnaire. In some aspects, the first questionnaire is adapted for completion by a third party. For example, the questionnaire can be intended to evaluate the clinical state of the beneficiary, but the questionnaire can be filled out by the user on behalf of the beneficiary. In such embodiments, the text of the questions in the questionnaire can be altered to reflect the differences in perspective between the user and the beneficiary (e.g., “I find it difficult to remember familiar people or events” can become “Finds it difficult to remember familiar people or events.”) Furthermore, questions concerning the internal mental state of the beneficiary can be removed or replaced by questions that describe externally observable behaviors: Does the patient fail to pay attention to details, make careless mistakes, or have difficulty focusing? Does the patient seem to not listen when spoken to directly, or look around when someone is speaking directly to him or her? In some aspects, the first questionnaire is adapted for use with military personnel: What is your current military status? What type of discharge did you receive from the military? What is your branch of service? In various aspects, the first questionnaire is adapted for use with patients older than a minimum age. For example, questions concerning substance abuse can be provided only to patients older than the minimum age of 13.

Central server 120 can perform the first automated assessment by communicating with a remote device to display questions and receive responses, consistent with disclosed embodiments. For example, central server 120 can perform the automated assessment as described with regard to FIGS. 2, 3A and 3B. For example, in some embodiments the first questionnaire can comprise a general questionnaire and one or more clinical state-specific questionnaires. Central server 120 can be configured to select questions for display from a pool of potential questions. In some embodiments, central server 120 can be configured to select questions based on the responses received to prior questions. For example, when central server 120 receives a response indicating that further questioning regarding a topic is warranted, central server 120 can be configured to ask additional questions concerning that topic. Likewise, when a response indicates that additional questions concerning a topic are unnecessary, central server 120 can be configured to refrain from asking additional questions concerning that topic. As an additional example, central server 120 can be configured to provide the question “Have you used a controlled substance within the last year?” In some embodiments, should the response be “No,” central server 120 can be configured to refrain from providing for display additional questions concerning the use of controlled substances. In various embodiments, should the response be “Yes,” central server 120 can be configured to provide for display additional questions concerning, for example, the frequency of use of controlled substances and the types of controlled substances used.

In some embodiments, central server 120 can be configured to diagnose the clinical state of the patient based in part on the responses to the first questionnaire. For example, as described in FIGS. 2, 3A, and 3B, central server 120 or remote device 110 can be configured to associate values with responses and weights with questions. In some aspects, a question can contribute to a clinical score based on the weight for the question and the value for the response. At least one of central server 120 or remote device 110 can be configured to diagnose the user or beneficiary as having (or not having) a clinical state based on the value of the clinical score and a predetermined value for a threshold. The steps of associating values and weights with responses and questions, of calculating clinical scores, and of diagnosing clinical states can be distributed between central server 120 and remote device 110 a number of ways. For example, remote device 110 can be configured to perform all of these steps and central server 120 can be configured to perform none of these steps. As another example, remote device 110 can be configured to perform some of these steps (e.g., calculation of clinical scores) and central server 120 can be configured to perform others (e.g., diagnosis of clinical states). As a further example, remote device 110 can be configured to perform none of these steps and central server 120 can be configured to perform all of these steps.

In some embodiments, central server 120 can be configured to store at least one of the responses to the questions, contributions of the questions to the clinical scores, the clinical scores, or the diagnosis in a medical record of the user or beneficiary. In this manner, a record of the assessment can be available to a reviewing healthcare worker, such as a clinician.

In some embodiments, process 600 can further include central server 120 receiving a confirmation of the diagnosis of the at least one clinical state. This confirmation can be received from a computing device operated by a healthcare provider, such as a clinician that has reviewed a record of the first assessment. In some embodiments, central server 120 can be configured to include this confirmation in a medical record of the user or beneficiary. Central server 120 can also receive an indication that a healthcare provider has rejected the diagnosis of the at least one clinical state. Central server 120 can be configured to indicate in a medical record of the user or beneficiary this rejection, for example by modifying the existing diagnosis or annotating the existing diagnosis.

In step 607, central server 120 can receive at least one second request to perform at least one follow-up automated assessment of the patient. This request can be received following completion of the first assessment of the user or beneficiary. As described above with regards to step 603, this request can be received from remote device 110, or from another computing device.

In step 609, central server 120 can perform at least one follow-up automated assessment of the patient. This automated assessment can be performed in response to the request of step 607. In some embodiments, central server 120 can be configured to perform the at least one follow-up assessment using at least one second questionnaire. In some embodiments, the at least one second questionnaire can include the same questions for each of the at least one follow-up automated assessments. This consistency can enable better tracking of the clinical state of the user or beneficiary. In some embodiments, the at least one second questionnaire can differ from the first questionnaire. For example, the first questionnaire can include questions that are not included in the at least one second questionnaire. As a further example, the first questionnaire can establish that certain clinical conditions are not present in the user or beneficiary. Questions concerning these clinical conditions may not be included in the at least one second questionnaire to reduce the burden of complying with the at least one follow-up assessment.

In some embodiments, the at least one follow-up assessment can be performed as described above with regards to FIGS. 2, 3A and 3B, similar to the first assessment. For example, when performing each follow-up assessment, central server 120 can be configured to provide questions for display. As least one of central server 120 or remote user 110 can be configured to perform at least one of associating responses with values, associating weights with questions, determining the contribution of questions to clinical scored based on the associated values and weights, or diagnosing clinical states based on the clinical scores and predetermined thresholds. Each follow-up assessment can include a pool of questions, and central server 120 can select questions for display based on the responses to previous questions. In some embodiments, process 600 can further include automatically updating the diagnosis of the user or beneficiary based on the at least one follow-up automated assessment.

The automated nature of system 100 allows follow-up assessments to be performed in a non-clinical setting, such as the home of a patient. Thus, the follow-up assessments can be performed using a remote device. Furthermore, the follow-up assessments can be performed by the user without any intervention or contact from a healthcare provider. This flexibility should enable greater compliance with the assessment regime than would be observed if the patient was required to return to a clinic or hospital for every follow-up assessment. In turn, this enables treatment regimens based on repeated follow-ups and careful tracking of the clinical condition of the patient. For example, multiple follow-ups can be performed. In some embodiments, these follow-ups can be performed at weekly, bi-weekly, or monthly intervals. For example, a follow-up assessment can be performed a week after the first assessment, and another follow-up assessment can be performed a week after this first follow-up assessment.

In some embodiments, as described above with regard to FIG. 2, system 100 can be implemented as an early warning system. For example, during or upon completion of the first assessment or any follow-up assessment, central server 120 can be configured to determine whether a potential harm criterion has been satisfied. For example, the potential harm criterion can be satisfied when a clinical score exceeds the predetermined potential harm threshold. As another example, the potential harm can depend upon the first automated assessment of the patient and the at least one follow-up automated assessment. For example, clinical scores for the first automated assessment can establish a baseline, and the at least one follow-up assessment can constitute a sufficient departure from the baseline to satisfy the potential harm criterion. For example, the first automated assessment, in combination with the at least one follow-up assessment, can establish a trend that indicates a worsening condition of the user or beneficiary. The potential harm can be harm to the patient (the user or beneficiary) or harm to another person. For example, the possibility of suicide or attacking another person. In some embodiments, central server 120 can be configured to send a message upon satisfaction of the potential harm criterion. This message can be sent to responder system 130, and can indicate that the patient may be at risk of harming themselves or another. This message can include an automatic email, automatic telephone call, automatic text message, or other automatically generated message known to one of skill in the art.

In some embodiments, system 100 can include at least one wearable device (e.g., a fitness tracker, pulse oximeter, etc.) configured to obtain activity data from the user or beneficiary. For example, in some embodiments, remote device 110 can be such a wearable device. In various embodiments, system 100 can include a wearable device distinct from remote device 110. In some aspects, this distinct wearable device can be configured to communicate with other components of system 100 through network 140. For example, this wearable device can be able to communicate using a cellular network. In various aspects, this wearable device can be configured to communicate with other components of system 100 using a wireless area network (e.g., Wi-Fi), personal area network (e.g., ZigBee, Bluetooth, Z-Wave etc.), or near-field network (e.g., RFID) according to a protocol known to one of skill in the art. In some embodiments, central server 120 can be configured to base assessments at least in part upon activity data of the patient (e.g., the user or beneficiary) received from the wearable device. For example, central server 120 can base at least one of the first automated assessment or the at least one or more follow-up assessments upon such activity data. The activity data can include physiological signals such as pulse rate, blood oxygenation, and EEG or ECG data. The activity data can include the number of steps taken in a time period; walking or running distance, pace, and duration; exercise data (e.g., manually entered exercise information or duration and intensity of exercise inferred from other recorded data); and sleep data (e.g., manually entered sleep information or duration and timing inferred from other recorded data such as heart rate and movement).

Following step 609, process 600 can end in step 611. As would be appreciated by one of skill in the art, the particular order and arrangement of steps disclosed in FIG. 6 is not intended to be limiting. For example, steps can be re-arranged or combined without departing from the envisioned embodiments. Steps can be divided into sub-steps that can be performed in a different order, or by different components of system 100. Furthermore, additional steps may be added to, or steps may be removed from, this process without departing from the envisioned embodiments. Like the first assessment, each follow-up assessment can be recorded in a medical record of the user or beneficiary. Similarly, a confirmation or a rejection of the diagnosis can be indicated in a medical record of the beneficiary or user.

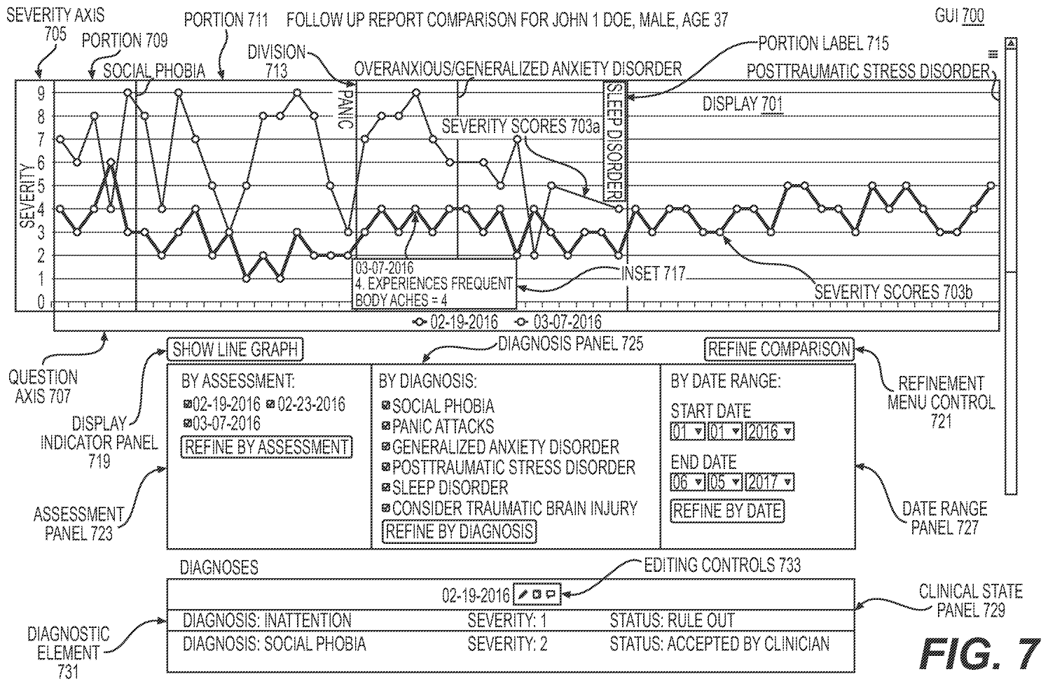

FIG. 7 depicts an exemplary graphical user interface (GUI 700) for displaying patient diagnostic information, consistent with disclosed embodiments. In some embodiments, central server 120 can be configured to provide instructions for displaying GUI 700. For example, central server 120 can provide such instructions to a computing device operated by a healthcare provider, such as a clinician. As an additional example, central server 120 can be configured to provide such instructions to remote device 110. In some embodiments, the computing device or remote device 110 can be configured to execute a client program. This client program can be configured to interact with central server 120 to provide at least some of the functionality of GUI 700. As would be appreciated by one of skill in the art, central server 120 and another computing device (e.g., a computing device operated by a healthcare provider or remote device 110) can be configured to interact to provide the functionality of GUI 700, and envisioned systems and methods are not limited to a particular set of interactions.

In some embodiments, GUI 700 comprises a display 701, display controls located below display 701, and a clinical-state panel 729. GUI 700 can be configured to show information from a medical record of a patient (e.g., a user or beneficiary). For example, display 701 can be configured to show results of assessments stored in the medical records. In some aspects, the display controls can enable selection of particular results for display. Clinical-state panel 729 can show the diagnoses associated with the patient. These diagnoses may have been automatically determined by system 100, as described above. Clinical-state panel 729 can provide functionality for confirming or rejecting, editing, or annotating these diagnoses. GUI 700 can be linked to the medical record for the patient, such that these confirmations or rejections, edits, or annotations are transferred to the medical record of the patient. In this manner, the medical record of the patient can be reviewed and optionally updated by a clinician or other healthcare provider.

Display 701 can be configured to depict severity scores (e.g., severity scores 703a and severity scores 703b) for one or more assessments of a patient (e.g., user or beneficiary), consistent with disclosed embodiments. The severity scores can correspond to responses to questions in one or more diagnostic questionnaires. For example, system 100 can be configured to associate a severity with a response to a question. In some aspects, this severity can be expressed as a category (e.g., “Absent,” “Mild,” “Moderate,” “Severe”) or a number within a range of numbers. As described above, system 100 can also be configured to associate a value with a response to a question. In some embodiments, system 100 can be configured to use the severity for the value associated with the response to the question. For example, the severity of a response to a question can be “4,” which can also be the value used to determine the contribution of the question to a clinical score. In various embodiments, system 100 can be configured to distinguish between the severity of a response and the value associated with the response for determining the contribution of the question to a clinical score. For example, responses to two different questions can both have the same severity, but the value for the responses can differ, when one response is more predictive of a clinical state than the other. Furthermore, by distinguishing between severity and value, GUI 700 can be configured to display severity scores for responses differing in predictive value on the same axis.

Display 701 can be linked to a medical record of a patient. For example, as the medical record is updated, display 701 can be updated. In some embodiments, a healthcare provider can therefore observe a user responding to questions in a questionnaire. In such embodiments,

remote device 110 can be configured to provide the responses to central server 120 (either individually or in batches). Central server 120 can be configured to store these responses in a medical record of the user or beneficiary. Because display 701 is linked to this medical record, central server 120 and the computing device displaying GUI 700 can then interact to display severity scores for these responses in display 701.

Display 701 can be configured to include severity axis 705, consistent with disclosed embodiments. Severity axis 705 can comprise indicators corresponding to the severity scores and arranged from least severity to greatest severity. For example, as shown in FIG. 7, when the severity scores are integers from 0 to 9, severity axis 705 can comprise indicators arranged from 0 to 9. As an additional example, severity axis 705 can comprise a color bar showing a range of colors transitioning from a first color that represents minimal severity to a second color that represents maximal severity (e.g., a color bar ranging from green through yellow to red).

Display 701 can include question axis 707, consistent with disclosed embodiments. Question axis 707 can comprise indicators corresponding to the questions in the one or more questionnaires. Question axis 707 can be arranged into multiple portions (e.g., portion 709 and portion 711) that each correspond to a clinical state (e.g., portion 709 can correspond to “social phobia”, portion 711 can correspond to “panic”). In some aspects, display 701 can depict labels indicating the clinical states corresponding to the multiple portions (e.g., portion label 715 indicates a portion of question axis 707 corresponding to the clinical state “Sleep Disorder”). In various aspects, display 701 can depict divider labels separating the multiple portions (e.g., division 713 indicates a separation between a portion for the “Panic” clinical state and a portion for the “Overanxious/Generalized Anxiety Disorder” clinical state).

Display 701 can be configured to depict an inset upon selection of a severity score. For example, as shown in FIG. 7, selection of one of severity scores 703b can cause display 701 to depict inset 717. As shown, inset 717 can be depicted in proximity to the selected severity score, and the connection between the selected severity score and inset 717 can be called out. Inset 717 can display information relating to the selected severity score. For example, inset 717 can display at least one of the date of the assessment, the text of the question corresponding to the severity score, the number of the question in the questionnaire, an identifier for the questionnaire (not shown in FIG. 7), or a response (e.g., a response value or text indicating the response).

The display controls arranged below display 701 can include display indicator control 719 and refinement menu control 721. Display indicator control 719 can be configured to control the representation of the severity scores in display 701. For example, display indicator control 719 can be selectable to switch the representation of the severity scores between line graphs corresponding to assessments (as shown in FIG. 7) and bar charts corresponding to assessments. Refinement menu control 721 can be selectable to cause GUI 700 to display at least one of assessment panel 723, diagnosis panel 725, or date range panel 727.

In some embodiments, assessment panel 723 displays selectable assessment controls. These assessment controls correspond to the plurality of assessments available for the patient. For example, when the patient has three available assessments, assessment panel 723 can display three selectable assessment controls. These assessment controls can be check boxes (as shown in FIG. 7), or a selectable dropdown menu, or other suitable controls known to one of skill in the art. As a non-limiting example, when using check-boxes for assessment controls, display 701 can be configured to depict only severity scores for the selected assessments. Assessment panel 723 can also comprise an assessment refinement control. Selecting the assessment refinement control can cause GUI 700 to update display 701 according to the current setting of the assessment controls. For example, to change the assessments displayed in display 701, a user could interact with GUI 700 to change the settings of the assessment controls and then select the assessment refinement control.

In some embodiments, diagnosis panel 725 displays selectable diagnosis controls. These diagnosis controls correspond to the plurality of clinical states potentially experienced by the patient. For example, as shown in FIG. 7, the patient may be diagnosed with “Social Phobia,” “Panic Attacks” and other listed clinical states. These diagnosis controls can be check boxes (as shown in FIG. 7), or a selectable dropdown menu, or other suitable controls known to one of skill in the art. As a non-limiting example, when using check-boxes for diagnosis controls, display 701 can be configured to depict only severity scores for questions concerning the selected clinical states. Diagnosis panel 725 can also comprise a diagnosis refinement control. Selecting the diagnosis refinement control can cause GUI 700 to update display 701 according to the current setting of the diagnosis controls. For example, to limit the severity scores displayed in display 701 of certain clinical states, a user could interact with GUI 700 to change the settings of the diagnosis controls and then select the diagnosis refinement control.

In some embodiments, date-range panel 727 displays selectable date-range controls. These date-range controls can include a start-date control indicating a start date and an end-date control indicating an end date. By interacting with these controls the user can modify the start date and the end date. In some embodiments, date-range panel 727 can include a selectable date-range refinement control. Selecting the date-range refinement control can cause GUI 700 to update display 701 according to the current settings of the start-date control and the end-date control. For example, a user could interact with GUI 700 to change the settings of the start-date control and the end-date control and then select the date-range refinement control to limit the severity scores displayed in display 701. In some embodiments, refinement by date range can cause display 701 to depict severity scores for diagnostic questions answered between the start date and the end date. In some embodiments, refinement by date range can cause display 701 to depict assessments started or completed between the start date and the end date.

Clinical-state panel 729 can be linked to a medical record of a patient. For example, as the medical record is updated, diagnosis panel 729 can be updated. Clinical-state panel 729 can be configured to allow confirming or rejecting, editing, and annotating of diagnosis for different clinical states. In some embodiments, a healthcare provider can therefore review the assessments using GUI 700 and edit the diagnosis using Clinical-state panel 729, thereby updating the medical records for the patient.

In some embodiments, Clinical-state panel 729 can include one or more diagnostic elements (e.g., diagnostic element 731). These diagnostic elements can summarize information about the patient. A diagnostic element can include an indication of a clinical state (e.g., “Diagnosis: Inattention”), an indication of the severity of this clinical state (e.g., “Severity: 1”) and a status of this diagnosis. The severity of a clinical state can depend upon a clinical score for the clinical state. In this manner, the severity of a clinical state differs from the severity scores for responses to questions depicted in display 701. The status of a diagnosis can include “Rejected” in which the diagnosis is rejected by a healthcare provider, “Ruled Out” in which some symptoms of a clinical state are present but not enough to form a diagnosis, and “Accepted” in which the diagnosis is confirmed by a healthcare provider. As would be understood by one of skill in the art, the particular text of the status indicator is not intended to be limiting (e.g., “Accepted” would be recognized as equivalent to “Accepted by the Clinician”). In some embodiments, each diagnostic element can also include a text field showing annotations associated with the clinical state (not shown in FIG. 7).

In some embodiments, clinical-state panel 729 can include editing controls 733, which can be used to enter an editing mode. While in the editing mode, one or more fields of the diagnostic elements can appear as changeable controls, pre-populated with the current information for those fields. For example, the severity indicator of diagnostic element 731 can become a severity control, such as a drop-down menu, showing a range of selectable severity options (e.g., a range of numbers). Likewise, the status indicator of diagnostic element 731 can become a status control, such as a drop-down menu, showing a range of selectable status options (e.g., “Accepted,” “Rejected,” Ruled-Out,” etc.) Re-selecting the editing controls can cause GUI 700 to exit this editing mode.

As would be appreciated by one of skill in the art, the specific arrangement of graphical user interface elements and functionality in FIG. 7 is not intended to be limiting. For example, graphical user elements can be added, removed, re-arranged or combined without departing from the envisioned embodiments. For example, additional graphical user elements can be added with additional functionality. As an additional example, the functionality of multiple graphical user interface elements can be combined into single elements. Furthermore, the graphical user interface functionality described can be divided in various ways between components of system 100.

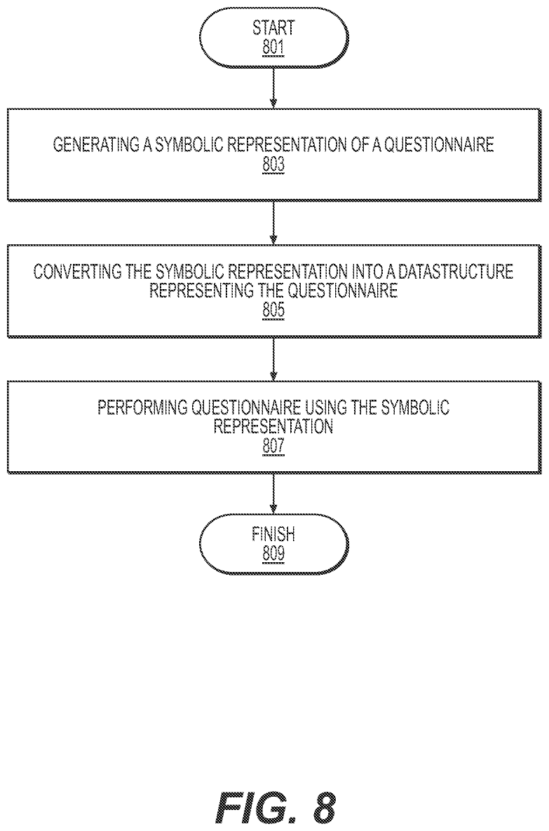

FIG. 8 depicts an exemplary process for generating questionnaires using a visual programming language. This visual programming language enables users unfamiliar with programming languages to construct automatic diagnostic questionnaires. Using this visual programming language, a questionnaire can be constructed by adding object to a window in a graphical user interface (the “algorithm blueprint”) and then connecting the objects, similar to other visual programming languages like LABVIEW by NATIONAL INSTRUMENTS. This visual representation of the questionnaire can be converted into instructions. These instructions can be saved in a format that evaluation software running on central server 120 or remote device 110 can understand. In this manner, rather than coding the questionnaires into the evaluation software, the evaluation software can be configured to accept instructions describing questionnaires. These instructions can be evaluated to diagnosis different clinical states of a user or beneficiary. In some embodiments, the questionnaire can concern at least one of suicidal ideation, depression, schizophrenia, traumatic brain injury, or executive function.

After starting in step 801, a computing device can be configured to provide a platform for creating a symbolic representation of a questionnaire. This symbolic representation can include question objects and answer objects, container object, and line objects that connect other objects. The computing device can be configured to generate a symbolic representation of a questionnaire based on input from a user.

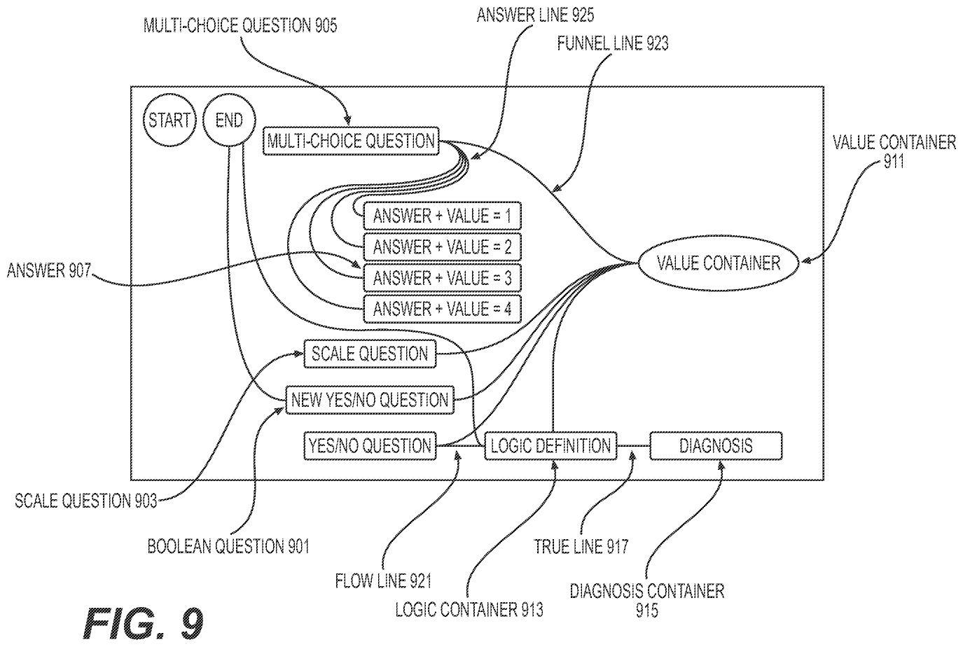

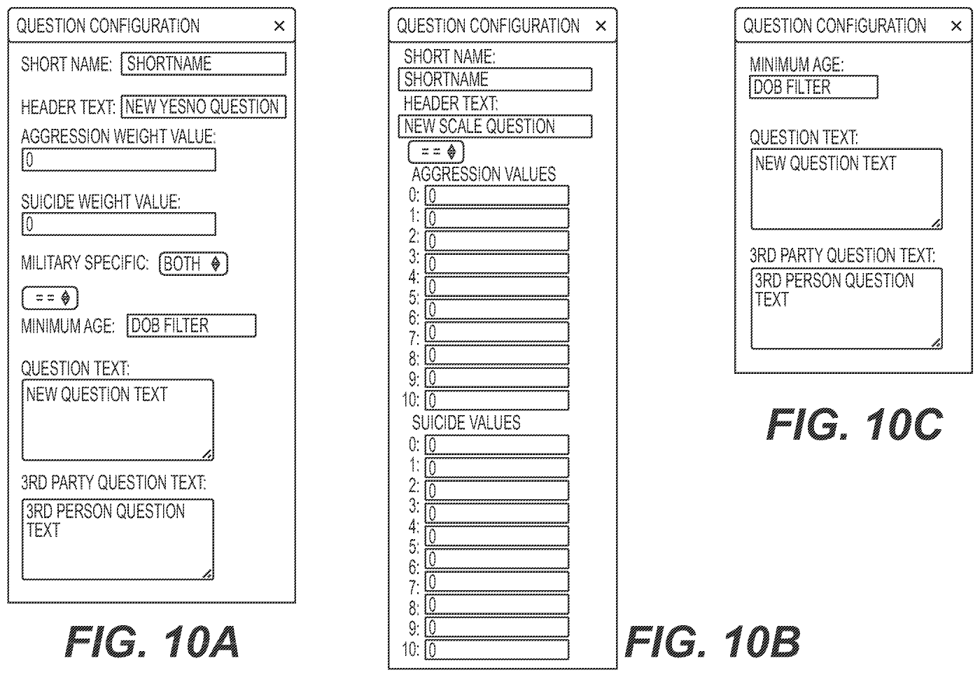

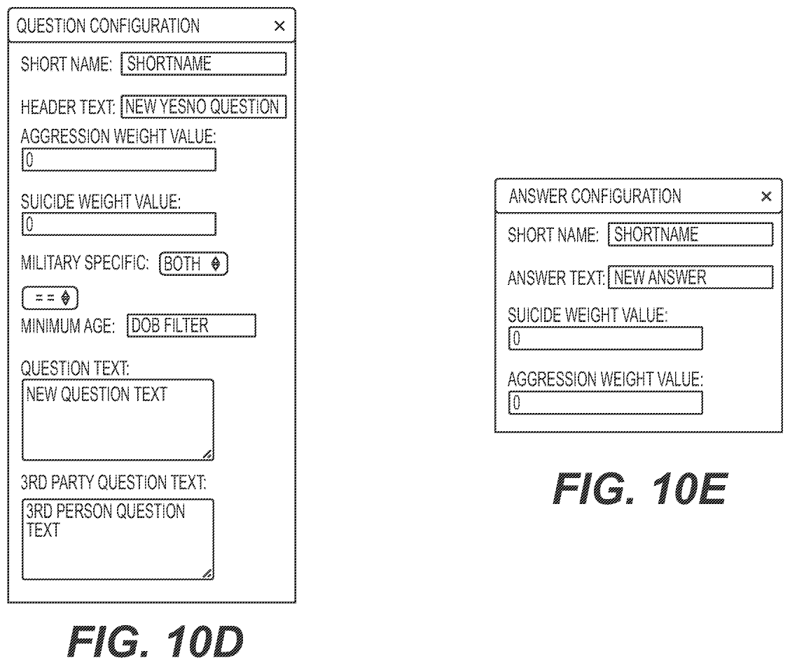

Question objects can include at least one of Boolean question objects, scale question objects, multi-choice question objects, or text question objects, consistent with disclosed embodiments. These question objects can be displayed as shapes in the algorithm blueprint. Boolean question objects can correspond to questions in a questionnaire having yes/no answers, consistent with disclosed embodiments. Boolean question objects can be configured with multiple fields. For example, a Boolean question object can be configured with a question text that comprises the question displayed to the user and one or more values for one or more different clinical states. For example, a Boolean question object can be configured with a value for aggression and a value for suicide. In some embodiments, a Boolean question object can include fields corresponding to at least one of a display name (for labeling the object in the algorithm blueprint), header text (for labeling the question in a diagnostic questionnaire), a military specific control selectable to indicate whether the question is for use in questionnaires adapted for military personnel, an age control, or a 3rd party question text. In some embodiments, the age control can specify an age criterion that must be satisfied to display the question. For example, this age control can comprise an operator for age and a specified age. The operator for age can specify that to be displayed the question the user or beneficiary must be greater than, equal to, or less than the specified age. The 3rd party question text can be displayed when a user is completing the questionnaire on behalf of a beneficiary. FIG. 9 depicts an exemplary Boolean question object (Boolean question 901) as shown in the algorithm blueprint. In some elements, the visual programming language can be configured to display an interface for editing this Boolean question object. For example, a user can select the Boolean question object to open an interface for editing the Boolean question object, as shown in FIG. 10A. This interface can permit editing of the fields of the Boolean question object.

Scale question objects can correspond to questions in a questionnaire having a range of values as responses, consistent with disclosed embodiments. FIG. 11A depicts a non-limiting example of a scale question. Scale question objects can be configured with multiple fields. For example, a scale question object can be configured with a question text that comprises the question displayed to the user and one or more sets of values for one or more different clinical states. As an example, a scale question can be configured with a set of values for aggression and a set of values for suicide. In some embodiments, a user can be provided with the option of selecting a severity in response to the scale question (e.g., selecting a number representing a severity from 0-10). The sets of values can map this severity to values for the one or more clinical states. For example, a severity of “2” can map to a value of “2” for aggression and a value of “2” for suicide, while a severity of “10” can map to a value of “10” for aggression and a value of “15” for suicide. In some embodiments, a scale question object can include fields corresponding to at least one of a display name (for labeling the object in the algorithm blueprint), header text (for labeling the question in a diagnostic questionnaire), a military specific control selectable to indicate whether the question is for use in questionnaires adapted for military personnel, an age control, or a 3rd party question text. In some embodiments, the age control can specify an age criterion that must be satisfied to display the question. For example, this age control can comprise an operator for age and a specified age. The operator for age can specify that to be displayed the question the user or beneficiary must be greater than, equal to, or less than the specified age. The 3rd party question text can be displayed when a user is completing the questionnaire on behalf of a beneficiary. FIG. 9 depicts an exemplary scale question (scale question 903) as shown in the algorithm blueprint. In some elements, the visual programming language can be configured to display an interface for editing this scale question object. For example, a user can select the scale question object to open an interface for editing the scale question object, as shown in FIGS. 10B and 10C. This interface can permit editing of the fields of the scale question object.

Multi-choice question objects can correspond to questions in a questionnaire having multiple choice answers, consistent with disclosed embodiments. Non-limiting examples of multi-choice questions include radio or check box questions (e.g., the questions depicted in FIGS. 11C, 11D, 12A and 12B). Multi-choice question objects can be configured with multiple fields. For example, a multi-choice question object can be configured with a question text that comprises the question displayed to the user. In some embodiments, a multi-choice object can include fields corresponding to at least one of a display name (for labeling the object in the algorithm blueprint), header text (for labeling the question in a diagnostic questionnaire), a military specific control selectable to indicate whether the question is for use in questionnaires adapted for military personnel, an age control, or a 3rd party question text. In some embodiments, the age control can specify an age criterion that must be satisfied to display the question. For example, this age control can comprise an operator for age and a specified age. The operator for age can specify that to be displayed the question the user or beneficiary must be greater than, equal to, or less than the specified age. The 3rd party question text can be displayed when a user is completing the questionnaire on behalf of a beneficiary. FIG. 9 depicts an exemplary Multi-choice question object (Multi-choice question 905) as shown in the algorithm blueprint. In some elements, the visual programming language can be configured to display an interface for editing this multi-choice question object. For example, a user can select the multi-choice question object to open an interface for editing the multi-choice question object, as shown in FIG. 10D. This interface can permit editing of the fields of the multi-choice question object.

Multi-choice objects can be connected with answer objects, consistent with disclosed embodiments. Given an algorithm blueprint for a questionnaire includes a multi-choice question object linked to answer objects, when the questionnaire is converted to instructions and executed by the software, the possible answers to the multi-choice question can be the linked answers.

These answer objects can be configured with multiple fields, including fields corresponding to at least one of a display name (for labeling the object in the algorithm blueprint), answer text shown to the user taking the questionnaire, or one or more values for one or more clinical conditions. For example, an answer object can be configured with a value for aggression and a value for suicide. FIG. 9 also depicts an exemplary answer object (answer object 907) as shown in the algorithm blueprint. In some elements, the visual programming language can be configured to display an interface for editing this answer object. For example, a user can select the answer object to open an interface for editing the answer object, as shown in FIG. 10E. This interface can permit editing of the fields of the answer object.

Text question objects can correspond to questions in a questionnaire having yes/no answers, consistent with disclosed embodiments. Text question objects can be configured with multiple fields. For example, a text question object can be configured with a question text that comprises the question displayed to the user and one or more values for one or more different clinical states. For example, a text question object can be configured with a value for aggression and a value for suicide. In some embodiments, a Boolean question object can include fields corresponding to at least one of a display name (for labeling the object in the algorithm blueprint), header text (for labeling the question in a diagnostic questionnaire), a military specific control selectable to indicate whether the question is for use in questionnaires adapted for military personnel, an age control, or a 3rd party question text. In some embodiments, the age control can specify an age criterion that must be satisfied to display the question. For example, this age control can comprise an operator for age and a specified age. The operator for age can specify that to be displayed the question the user or beneficiary must be greater than, equal to, or less than the specified age. The 3rd party question text can be displayed when a user is completing the questionnaire on behalf of a beneficiary.

Container objects can include at least one of value containers, logic containers, or diagnosis containers, consistent with disclosed embodiments. These container objects can be displayed as shapes in the algorithm blueprint.

Value container objects can represent functions performed on values received from other objects, consistent with disclosed embodiments. In some embodiments, a value container object can receive values from objects connected to the value container object by funnel lines. The value container object can be configured to perform the specified function on the received values. FIG. 9 depicts an exemplary value container object (value container 911) as shown in the algorithm blueprint.

Logic container objects can represent logical operations performed on values received from value objects, consistent with disclosed embodiments. In some embodiments, a logic container object can receive values from value objects connected to the logic container object by funnel lines. The logic container object can be configured to perform the specified logical operation on the received values. FIG. 9 depicts an exemplary logic container object (logic container 913) as shown in the algorithm blueprint.

Diagnosis container objects can represent diagnoses reached or excluded by system 100, consistent with disclosed embodiments. For example, a diagnosis container can be associated with a diagnosis of a particular clinical state (e.g., “suicide”). When a diagnosis is reached by a “true” or “false” line then the diagnosis can be triggered and added to a report generated upon execution of the questionnaire. In some embodiments, a medical record of the user or beneficiary can be modified based on the triggered diagnosis. Depending on whether the diagnosis is reached by a “true” or “false” line, the diagnosis can be either indicated or excluded. FIG. 9 depicts an exemplary diagnosis container object (diagnosis container 915) as shown in the algorithm blueprint.

Line objects can include at least one of true/false lines, flow lines, funnel lines, and answer lines, consistent with disclosed embodiments. These line objects can be displayed as lines connecting objects in the algorithm blueprint. In some embodiments, a questionnaire can be ordered such that an object or container is not evaluated until all of the lines leading into that question have been evaluated.

FIG. 9 depicts exemplary true line 917 and false line 919. True lines and false lines can connect Boolean question objects to other objects and logic container objects to other objects, consistent with disclosed embodiments. When a Boolean question object or a logic container object is true, then the questionnaire can include the object or objects connected to the Boolean question object or logic container object by a true line. Likewise, when a Boolean question object or a logic container object is false, then the questionnaire can include the object or objects connected to the Boolean question object or logic container object by a false line. As described above, a diagnosis can be indicated or excluded based on whether the diagnosis was reached using a true or false line.

FIG. 9 depicts exemplary flow line 921. Flow lines can determine which direction the algorithm will follow, consistent with disclosed embodiments. As described above, the questionnaire can be ordered such that objects and containers are not executed unless all of the input lines to the object or containers have been executed. Adding flow lines connecting objects can therefore establish an execution order for those objects. For example, connecting a first object to a second object using a flow line can ensure that the first object is answered prior to the second object.

FIG. 9 depicts exemplary funnel line 923, consistent with disclosed embodiments. Funnel lines can be used to funnel values into value containers and logic containers. In some embodiments, funnel lines can perform functions on received values. For example, a funnel line can be configured to threshold values generated by objects or containers connected using the funnel line. Alternatively or additionally, the objects or containers can be configured to only pass a value using the funnel lines when criteria are satisfied. For example, Boolean question objects can be configured to pass positive values when selected answer to the Boolean question is “Yes.” As an additional example, scale question objects can be configured to only pass positive values when the selected severity has a value greater than “4.” As a further example, multi-choice objects can be configured to always pass a value when a connected answer is selected.

FIG. 9 also depicts exemplary answer line 925, consistent with disclosed embodiments. Answer lines can be used to specify the answer objects corresponding to a multi-choice object. For example, a multi-choice object can be linked to answer objects using answer lines.

In some embodiments, a questionnaire can further include a start object and an end object. The start object can be used to define the beginning of the questionnaire. The end object can be used to define the end of the questionnaire. In some embodiments, the end object can be connected to true or false lines, as shown in FIG. 9. When the end object is reached by a true or false line, the questionnaire can be configured to end, regardless of the number of questions remaining.

In some embodiments, after constructing the symbolic representation of a questionnaire in step 803, the computing device can be configured to convert the symbolic representation of a questionnaire into at least one data structure representing the questionnaire. The computing device can be configured to perform this conversion in response to input received from a user. This data structure can be configured to be useable by software running on central server 120. In some embodiments, this data structure can comprise an XML array. In certain aspects, this XML array can include seven sections.

The first section can contain information related to the questions asked in the questionnaire, consistent with disclosed embodiments. This section can be an array, with entries comprising question objects. These entries can include multiple fields. An example of the fields stored for a Boolean question is provided:

<index0 type=“string”>Short Name for display within algorithm blueprint
</index0>
<index1 type=“string”>Type of Question</index1>
<index2 type=“string”>Text to display on the final report</index2>
<index3 type=“string”>Military Specific</index3>
<index4 type=“string”>Minimum Age for question to display</index4>
<index5 type=“string”>Question displayed to the end user(First
Person)</index5>
<index6 type=“string”> Question displayed to the end user(Third Party)
</index6>
Note: Adding “hide” will skip this question for third party assessments.
<index7 type=“string”>Horizontal position within algorithm blueprint
</index7>
<index8 type=“string”>Vertical position within algorithm blueprint</index8>
<index9 type=“string”>Unique identifier</index9>
<index10 type=“string”>Suicide Weight Value</index10>
<index11type=“string”>Aggression Weight Value</index11>

The second section can contain information related to the answers for multi-choice question object, consistent with disclosed embodiments. This section can be an array, with entries comprising answers corresponding to answer objects in the algorithm blueprint.

<index0 type=“string”>Answer Type</index0>
<index1 type=“string”>Answer Text</index1>
<index2 type=“string”> Suicide Weight Value </index2>
<index3 type=“string”> Horizontal position within algorithm blueprint
</index3>
<index4 type=“string”> Vertical position within algorithm blueprint </index4>
<index5 type=“string”> Unique identifier </index5>
<index6 type=“string”> Aggression Weight Value </index6>

The third section contains information related to line objects, consistent with disclosed embodiments. This section can be an array, with entries comprising lines corresponding to line objects in the algorithm blueprint.

<index0 type=“string”>Unique Identifier where line begins</index0>
<index1 type=“string”> Unique Identifier where line ends </index1>
<index2 type=“string”>Color of line in algorithm blueprint (hex
code)</index2>
<index3 type=“string”>Unused</index3>
<index4 type=“string”>Type of line</index4>

The fourth section contains information related to diagnosis objects, consistent with disclosed embodiments. This section can be an array, with entries comprising diagnosis corresponding to diagnosis objects in the algorithm blueprint.

<index0 type=“string”>Short Name of diagnosis</index0>
<index1 type=“string”>Full name of diagnosis </index1>
<index2 type=“string”> Diagnosis code</index2>
<index3 type=“string”> Horizontal position within algorithm blueprint
</index3>
<index4 type=“string”> Vertical position within algorithm blueprint </index4>
<index5 type=“string”> Unique identifier </index5>

The fifth section contains information related to value container objects, consistent with disclosed embodiments. This section can be an array, with entries comprising value containers corresponding to value container objects in the algorithm blueprint.

<index0 type=“string”>Display text for algorithm blueprint </index0>
<index1 type=“number”>Unused</index1>
<index2 type=“string”> Horizontal position within algorithm blueprint
</index2>
<index3 type=“string”> Vertical position within algorithm blueprint </index3>
<index4 type=“string”> Unique identifier </index4>
<index5 type=“string”>Container Type</index5>

The sixth section contains information related to logic container objects, consistent with disclosed embodiments. This section can be an array, with entries comprising logic containers corresponding to logic container objects in the algorithm blueprint.

<index0 type=“string”>Display name for algorithm blueprint </index0>
<index1 type=“string”>Logic operator (>,>=)</index1>
<index2 type=“string”>Value</index2>
<index3 type=“string”> Horizontal position within algorithm blueprint
</index3>
<index4 type=“string”> Vertical position within algorithm blueprint </index4>
<index5 type=“string”> Unique identifier </index5>

The seventh array can be a legacy array. This optional legacy array can be used to define the beginning and end of the algorithm. For example, this array can be used to terminate the questionnaire when a specific answer forces the questionnaire to end. This array can be static and identical across all questionnaires.

<index6 type=“array”>
<index0 type=“array”>
<index0 type=“string”>Start</index0>
<index1 type=“number”>0</index1>
<index2 type=“string”>150</index2>
<index3 type=“string”>50</index3>
<index4 type=“string”>bda7d89a-e240-466f-d328-6543b0780717</index4>
</index0>
<index1 type=“array”>
<index0 type=“string”>End </index0>
<index1 type=“number”>0</index1>
<index2 type=“string”>250</index2>
<index3 type=“string”>50</index3>
<index4 type=“string”>bde7d89a-e240-466f-d328-1533b0780717</index4>
</index1>
</index6>

After step 805, at least one of central server 120 or remote device 110 can be configured to receive the data structures representing the questionnaire. Central server 120 or remote server 110 can be configured to execute the received questionnaire in response to a request, as described above with regard to FIGS. 2, 3A and 3B. In some embodiments, central server 120 or remote server 110 can be configured to execute the received questionnaire using a scripting language, such as Javascript or PHP: Hypertext Preprocessor, to generate instructions for displaying the questionnaire, according to methods known to one of skill in the art.

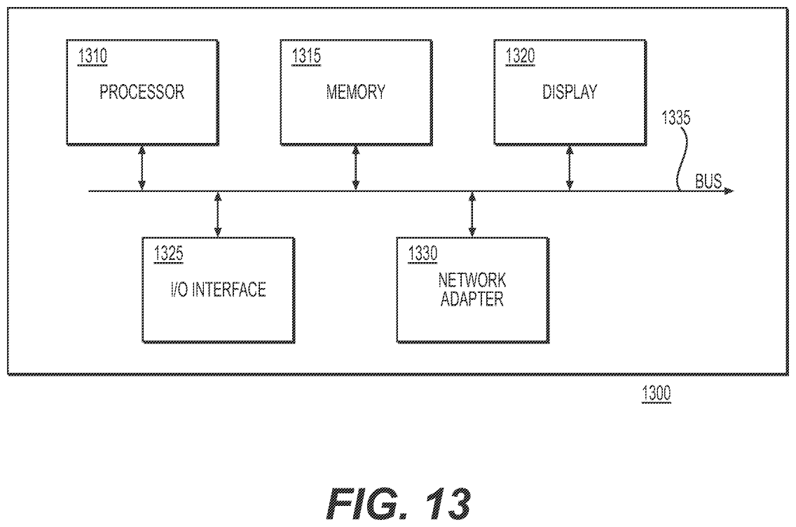

FIG. 13 depicts a schematic of exemplary computing system 1300 for performing the envisioned systems and methods, consistent with disclosed embodiments. In some embodiments, computing system 1300 includes a processor 1310, memory 1315, display 1320, I/O interface(s) 1325, and network adapter 1330. These units may communicate with each other via bus 1335, or wirelessly. The components shown in FIG. 13 may reside in a single device or multiple devices.

Consistent with disclosed embodiments, processor 1310 may comprise a central processing unit (CPU), graphical processing unit (GPU), or similar microprocessor having one or more processing cores. Computing system 1300 may include one or more processors 1310 and may further operate with one or more other processors that are remote with respect to processors 1310. Memory 1315 may include non-transitory memory containing non-transitory instructions, such as a computer hard disk, random access memory (RAM), removable storage, or remote computer storage. In some aspects, memory 1315 may be configured to store data and instructions, such as software programs. For example, memory 1315 may be configured to store data and instructions. In some aspects, processor 1310 may be configured to execute non-transitory instructions and/or programs stored on memory 1315 to configure computing system 1300 to perform operations of the disclosed systems and methods. In various aspects, as would be recognized by one of skill in the art, processor 1310 may be configured to execute non-transitory instructions and/or programs stored on a remote memory to perform operations of the disclosed systems and methods.

Display 1320 may be any device which provides a visual output, for example, a computer monitor, an LCD screen, etc. I/O interfaces 1325 may include hardware and/or a combination of hardware and software for communicating information to computing system 1300 from a user of computing system 1300, such as a keyboard, mouse, trackball, audio input device, touch screen, infrared input interface, or similar device. Network adapter 1330 may include hardware and/or a combination of hardware and software for enabling computing system 1300 to exchange information using external networks, such as network 140. For example, network adapter 1330 may include a wireless wide area network (WWAN) adapter, a Bluetooth module, a near field communication module, or a local area network (LAN) adapter.

Other embodiments will be apparent to those skilled in the art from consideration of the specification and practice of the disclosed embodiments disclosed herein. It is intended that the specification and examples be considered as exemplary only, with a true scope and spirit of the disclosed embodiments being indicated by the following claims. Furthermore, although aspects of the disclosed embodiments are described as being associated with data stored in memory and other tangible computer-readable storage mediums, one skilled in the art will appreciate that these aspects can also be stored on and executed from many types of tangible computer-readable media, such as secondary storage devices, like hard disks, floppy disks, or CD-ROM, or other forms of RAM or ROM. Accordingly, the disclosed embodiments are not limited to the above described examples, but instead is defined by the appended claims in light of their full scope of equivalents.

Moreover, while illustrative embodiments have been described herein, the scope includes any and all embodiments having equivalent elements, modifications, omissions, combinations (e.g., of aspects across various embodiments), adaptations or alterations based on the present disclosure. The elements in the claims are to be interpreted broadly based on the language employed in the claims and not limited to examples described in the present specification or during the prosecution of the application, which examples are to be construed as non-exclusive. Further, the steps of the disclosed methods can be modified in any manner, including by reordering steps or inserting or deleting steps. It is intended, therefore, that the specification and examples be considered as example only, with a true scope and spirit being indicated by the following claims and their full scope of equivalents.

1.-23. (canceled)

24. A system for patient evaluation comprising,
a mobile device including at least one mobile device processor and at least one non-transitory memory containing mobile device instructions that when executed by the at least one mobile device processor cause the mobile device to perform mobile device operations including:
conducting an automated assessment that detects at least one of traumatic brain injury or concussion, the automated assessment including in part displaying a clinical condition questionnaire, and receiving responses to the clinical condition questionnaire from the patient, and providing a record of the automated assessment of the patient to a central server; and
wherein the central server including at least one central server processor and at least one non-transitory memory containing central server instructions that when executed by the at least one central server processor cause the central server to perform central server operations including:
receiving the record of the automated assessment of the patient, receiving a request to determine a clinical state of a patient, diagnose the clinical state of the patient based on the received record of the automated assessment and stored records of prior automated assessments, and
providing an indication of the clinical state of the patient.

25. The system for patient evaluation of claim 24, wherein at least one of the mobile device operations or the central server operations further comprises:
calculating at least one of a traumatic brain injury score or concussion score based on values for the responses to the clinical condition questionnaire; and
determining the clinical state of the patient based in part on the at least one of the traumatic brain injury score or the concussion score.

26. The system for patient evaluation of claim 25, wherein the clinical state of the patient is determined based in part on a comparison of the at least one of the traumatic brain injury score or the concussion score to previously calculated scores.

27. The system for patient evaluation of claim 24, wherein the mobile device operations further include displaying the indication of the clinical state of the patient upon completion of the automated assessment.

28. An automated assessment system, comprising at least one processor; and at least one non-transitory computer readable medium containing instructions that, when executed by the at least one processor, cause the automated assessment system to perform operations including:
receiving, from a remote device, a first request to perform an automated assessment of a patient;
performing a first automated assessment of the patient using a first questionnaire to determine a potential clinical state of the patient in response to the first request;
receiving, from the remote device after determining the potential clinical state of the patient, at least one second request to perform at least one follow-up automated assessment of the patient; and
performing the at least one follow-up automated assessment of the patient using at least one second questionnaire in response to the at least one second request.

29. The automated assessment system of claim 28, wherein the operations further comprise providing an invitation to the patient, and wherein the first request to perform the automated assessment is received in response to the invitation.

30. The automated assessment system of claim 29, wherein the invitation comprises an email including registration instructions.

31. The automated assessment system of claim 28, wherein the first questionnaire is adapted for completion by a third party.

32. (canceled)

33. (canceled).

34. The automated assessment system of claim 28, wherein performing the first automated assessment of the patient using the first questionnaire comprises:
communicating with the remote device to display questions and receive responses, wherein at least one question is displayed based on at least one response to at least one previously displayed question; and
diagnosing the potential clinical state of the patient based in part on the responses to the first questionnaire.

35. The automated assessment system of claim 34, wherein the operations further comprise automatically updating the diagnosed potential clinical state of the patient based on the at least one follow-up automated assessment.

36. The automated assessment system of claim 34, wherein determining the potential clinical state of the patient comprises calculating, using the remote device, a clinical state score based on values for the received responses; and wherein the potential clinical state is determined based in part on the clinical state score.

37. The automated assessment system of claim 28, wherein the operations further comprise adding a result of the first automated assessment to an electronic medical record of the patient.

38. The automated assessment system of claim 28, wherein the operations further comprise receiving a confirmation of the at least one of the potential clinical state from a healthcare provider.

39. The automated assessment system of claim 28, wherein the first automated assessment and a first follow-up automated assessment of the at least one follow-up automated assessment are separated by at least a week.

40. The automated assessment system of claim 28, wherein the at least one follow-up automated assessment comprises communicating with the remote device to complete a questionnaire dependent on the potential clinical state.

41. The automated assessment system of claim 28, wherein the at least one follow-up automated assessment is performed in a non-clinical setting.

42. The automated assessment system of claim 28, wherein the operations further comprise sending a message upon satisfaction of a potential harm criterion.

43. The automated assessment system of claim 42, wherein the potential harm criterion depends upon the first automated assessment of the patient and the at least one follow-up automated assessment.

44. The automated assessment system of claim 28, wherein the at least one follow-up automated assessment depends at least in part upon activity data received from a wearable device of the patient.

45. The automated assessment system of claim 44, wherein the activity data comprises at least one of exercise data or sleep data.

46.-70. (canceled).
